(19) United States
(12) Patent Application Publication
(10) Pub. No.: US 2002/0151477 A1
(43) Pub. Date: Oct. 17, 2002

(54) Nuclear receptor coactivator

(76) Inventors: Jan-Ake Gustafsson, Stockholm (SE); Francoise Caira, Aubiere (FR); Per Antonsson, Stockholm (SE); Eckardt Treuter, Huddinge (SE)

Correspondence Address: Docketing Clerk, Intellectual Property Law Section, Wiggin & Dana, One Century Tower, New Haven, CT, 06508-1832, US

(21) Appl. No.: 09/735,367

(22) Filed: Dec. 12, 2000

Related U.S. Application Data
(60) Provisional application No. 60/174,544, filed on Jan. 5, 2000.

(51) Int. Cl. A61K 38/17; C07K 14/705; C07H 21/04; C12P 21/02; C12N 5/06

(52) U.S. Cl. 514/12; 435/69.1; 435/325; 435/320.1; 530/350; 536/23.5

(57) ABSTRACT

This invention relates to a new nuclear receptor coactivator RAP250 and to its uses.

This invention relates to a new nuclear receptor coactivator RAP250 and to its uses.

The nuclear hormone receptor (NR) superfamily is a large group of structurally related transcription factors that regulate target gene transcription in response to ligands. The complex genetic programs regulated by NRs include biological processes such as growth, cell differentiation and homeostasis (Mangelsdorf, D. J., et al (1995) Cell 83: 835-839). They can be divided into several subfamilies on the basis of characteristics such as dimerization status, nature of the ligand or structure of the DNA response element. NRs are characterized by a common domain structure, including a highly variable N-terminal region that contains a constitutive activation function (AF-1), a highly conserved DNA binding domain (DBD) responsible for recognition of specific DNA response elements and a conserved multifunctional C-terminal domain required for ligand binding (LBD), dimerization and ligand-dependent transactivation function (AF-2) (Mangelsdorf, D. J., and Evans, R. M. (1995) Cell 83: 841-850). The liganded NRs bind to their cognate hormone response elements, located in the promoter or enhancer regions of target genes, and stimulate transcriptional activation by transmitting signals to the transcriptional machinery via direct protein-protein interactions (Beato, M., and Sanchez-Pacheoc, A, (1996) Endocr. Rev. 17: 587-609; Hadzic, E., et al (1995) Mol. Cell. Biol. 15: 4507-4517; Schulman, I. G., et al (1995) Proc. Natl. Acad. Sci. USA 92: 8288-8292). In addition, another class of proteins, called coactivators, are recruited and serve as bridging molecules between the transcription initiation complex and NRs (for reviews see Glass, C. K., et al (1997) Curr. Opin. Cell. Biol. 9: 222-232 and Shibata, H., et al (1997) Recent Prog. Horm. Res. 52: 141-164). Most of the coactivators interact with the AF-2 domain of nuclear receptors through one or several LxxLL motifs called NR-boxes (Le Douarin, B., et al (1996) EMBO J. 15: 6701-6715; Torchia, J., et al (1997) Nature 387: 677-684; Heery, D. M. et al (1997) Nature 387: 733-736; McInerney, E. M. et al (1998) Genes & Dev. 12: 3357-3368; Shiau, A. K., et al (1998) Cell 95: 927-937). Bona fide AF-2 coactivators include the three related members of the p160/SRC family as well as the cointegrators CBP/p300 (for review, see Glass, C. K., et al, supra). Since these coactivators possess intrinsic histone acetyltransferase (HAT) activity and function in complex with other acetyltransferases such as P/CAF, it has been proposed that functional connections exist between NR activation and the histone acetylation status. Evidence for the existence of NR-coactivator complexes came from biochemical studies identifying the TRAP/DRIP complex (Fondell, J. D., et al (1998) Cell 95: 927-937; Fondell, J. D., et al (1999) Proc. Natl. Acad. Sci. USA 96, 1959-1964; Rachez, C., et al (1998) Genes & Dev. 12: 1781-1800; Rachez, C., et al (1999) Nature 398: 824-828) which may function more directly through contacts to the basal machinery. In addition to coactivators, other AF-2 binding proteins such as RIP140 (Cavaillès, V., et al (1995) EMBO J. 14: 3741-3751; Treuter, E., et al (1998) Endocrinol. 12: 864-881) or the nuclear orphan receptor SHP (Johansson, L., et al (1999) J Biol. Chem. 274: 345-353) may serve important regulatory functions by inhibiting NR activation.

To identify new potential coactivators, a mouse embryo cDNA library was screened using the yeast two-hybrid system with PPARα as bait. Here, we report the cloning and characterization of RAP250, a new nuclear receptor coactivator. Interestingly, this 250 kDa protein shows no homology to any described coactivators, except for the presence of an NR-box. Nevertheless, we show that RAP250 possesses an intrinsic activation domain and functions as a coactivator for a large number of hormone-dependent nuclear receptors. Interestingly, RAP250 shows a preferential affinity for the subtype β of estrogen receptor (ERβ) and unlike most of the coactivators identified to date, shows a tissue-specific expression, with high mRNA levels in brain and reproductive organs such as testis, ovary, prostate which are known as ER-rich tissues. Thus, its particular tissue distribution, its lack of related sequence to any other coactivators and its stronger affinity of ERβ make RAP250 a unique member among the coactivators described to date.

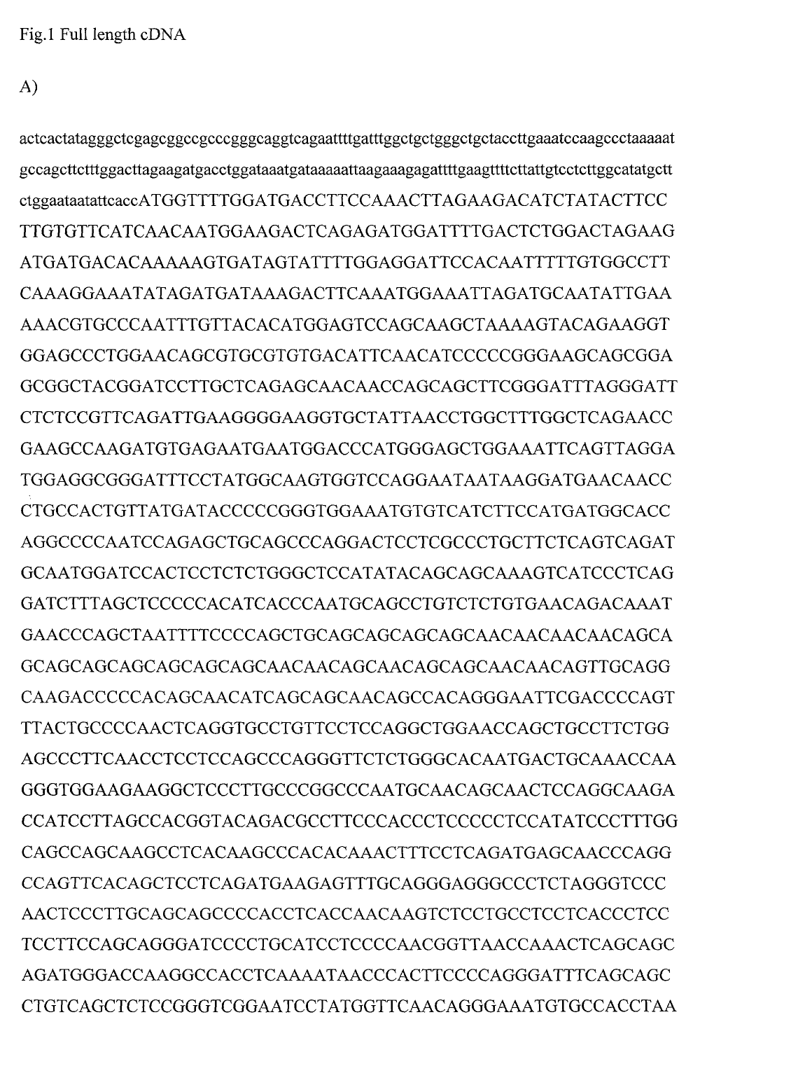

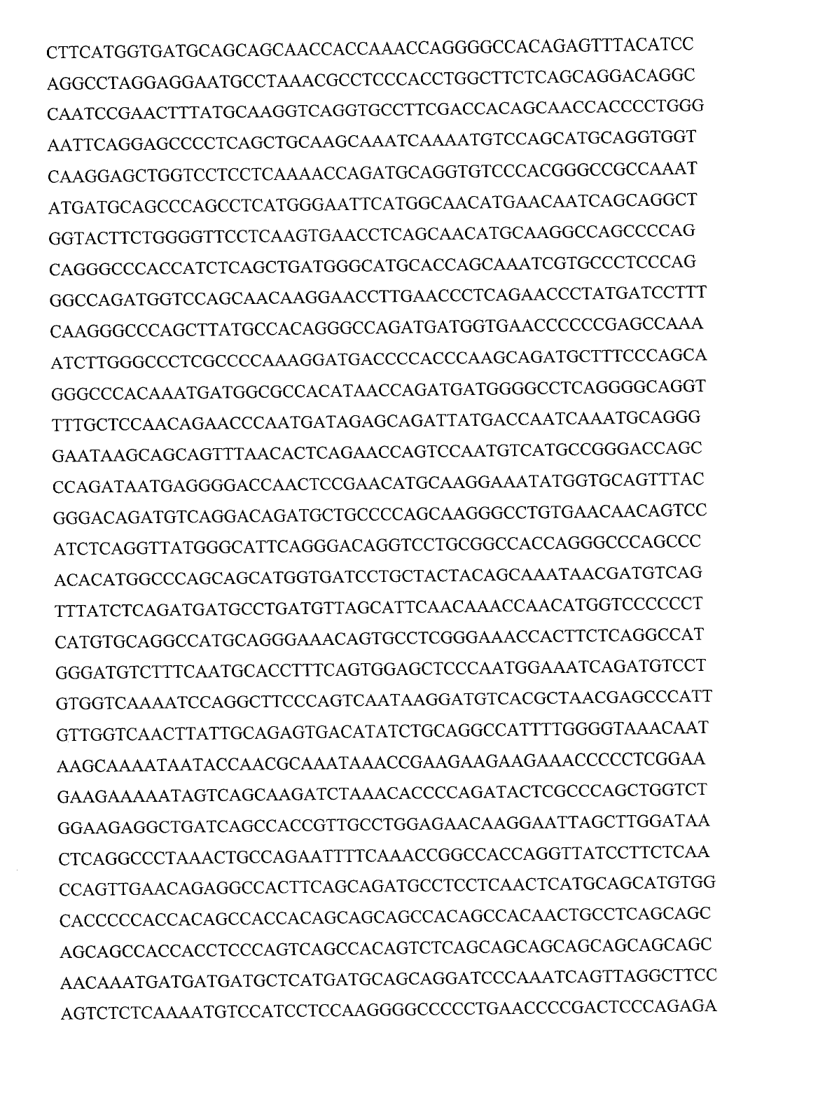

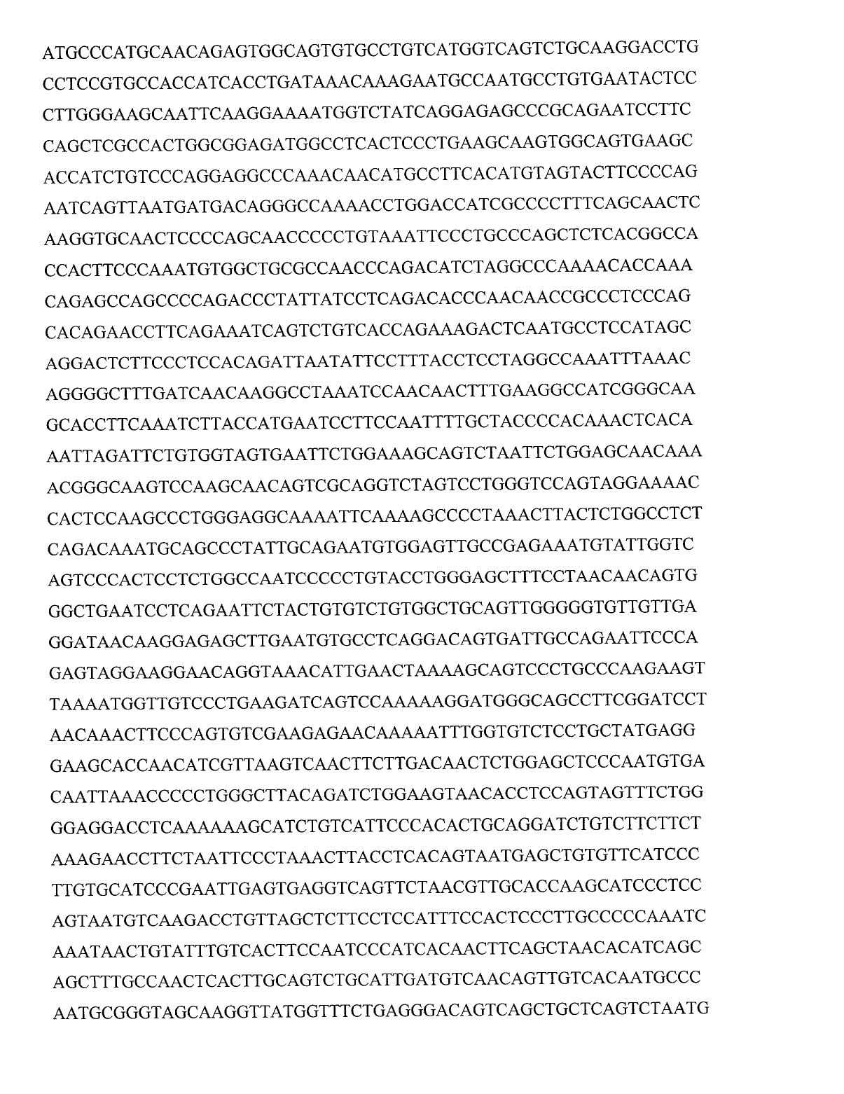

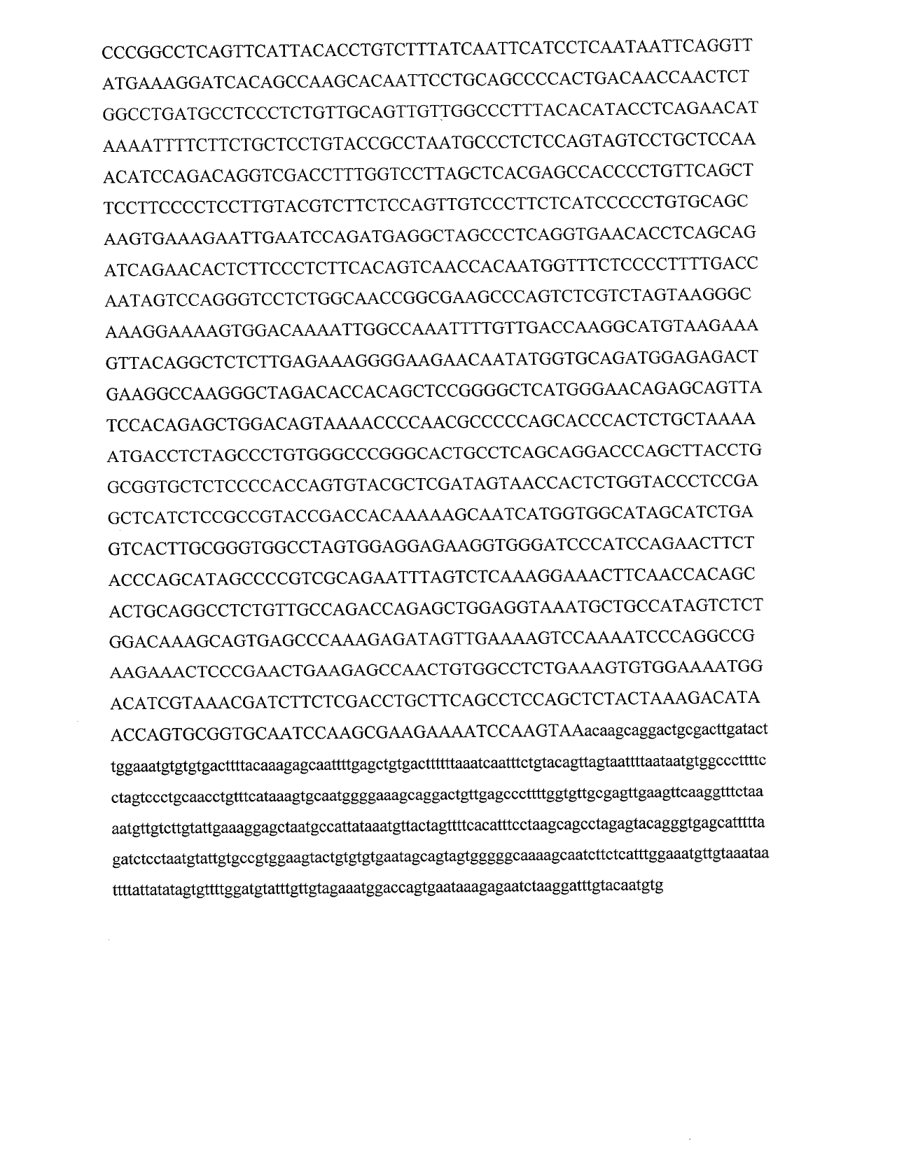

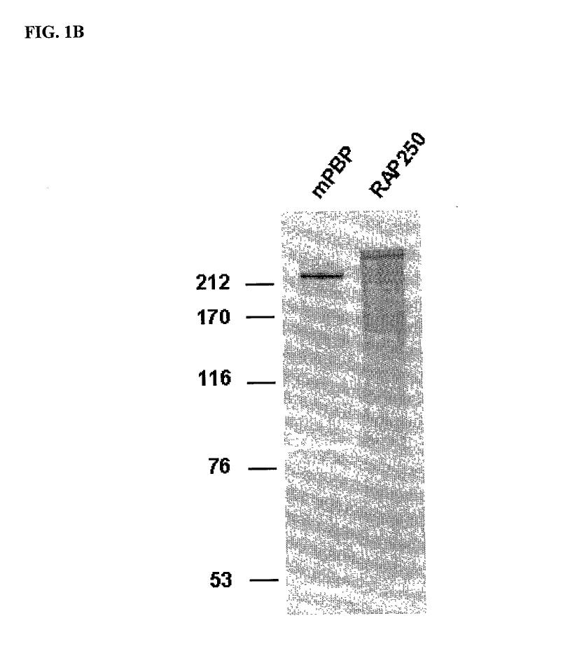

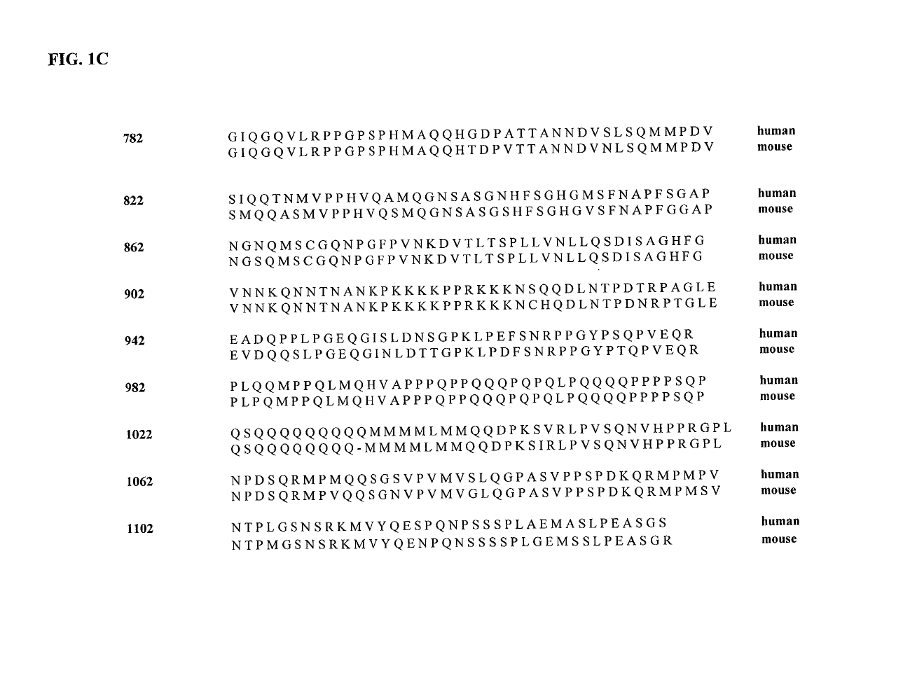

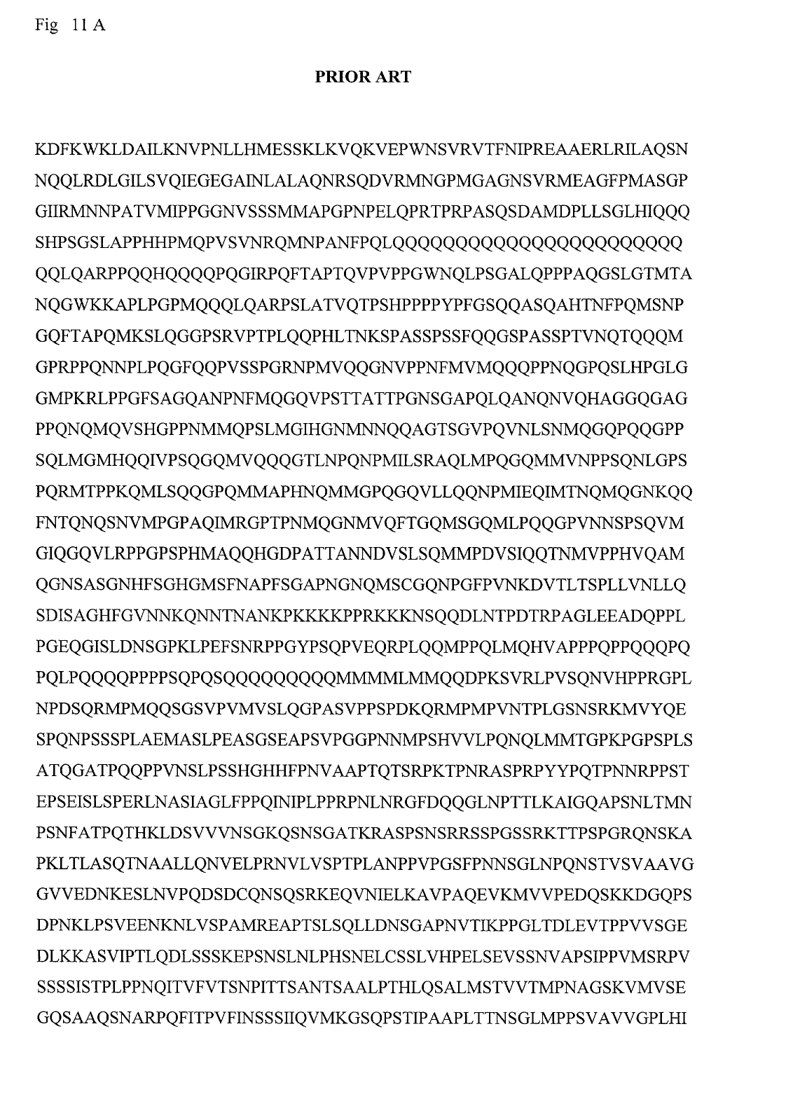

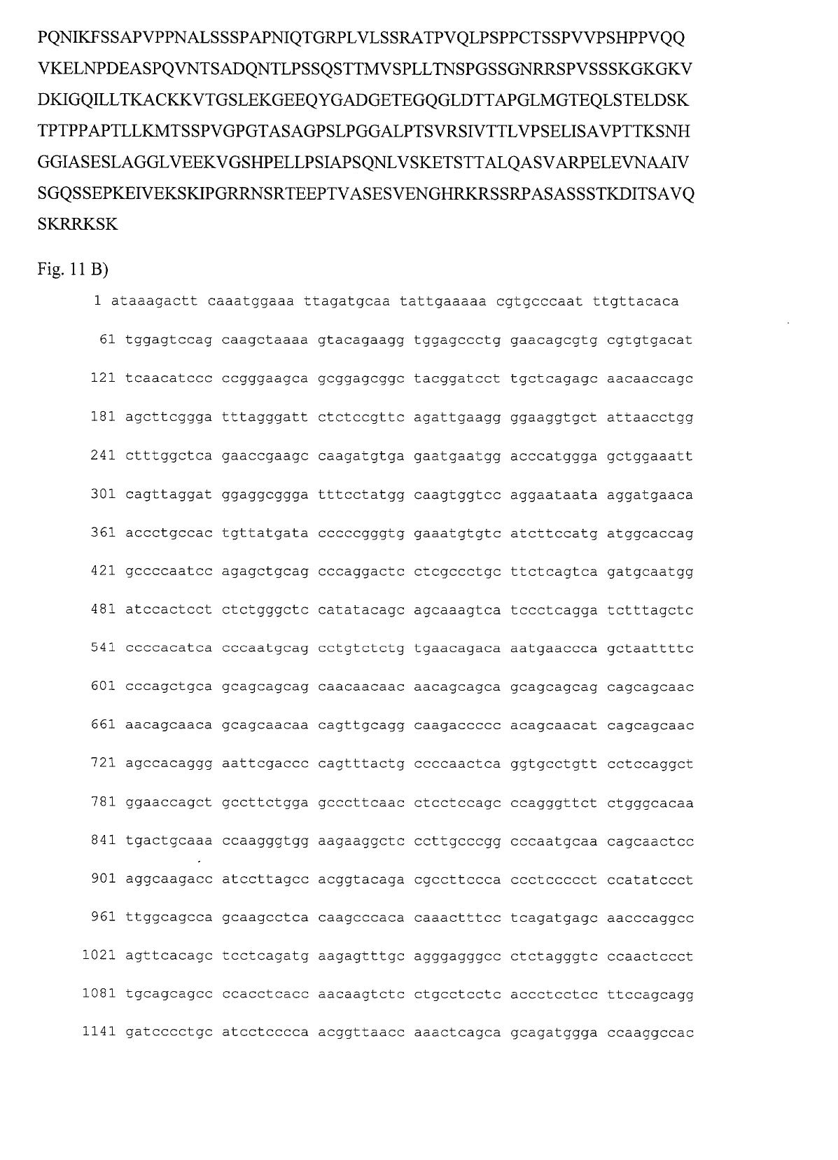

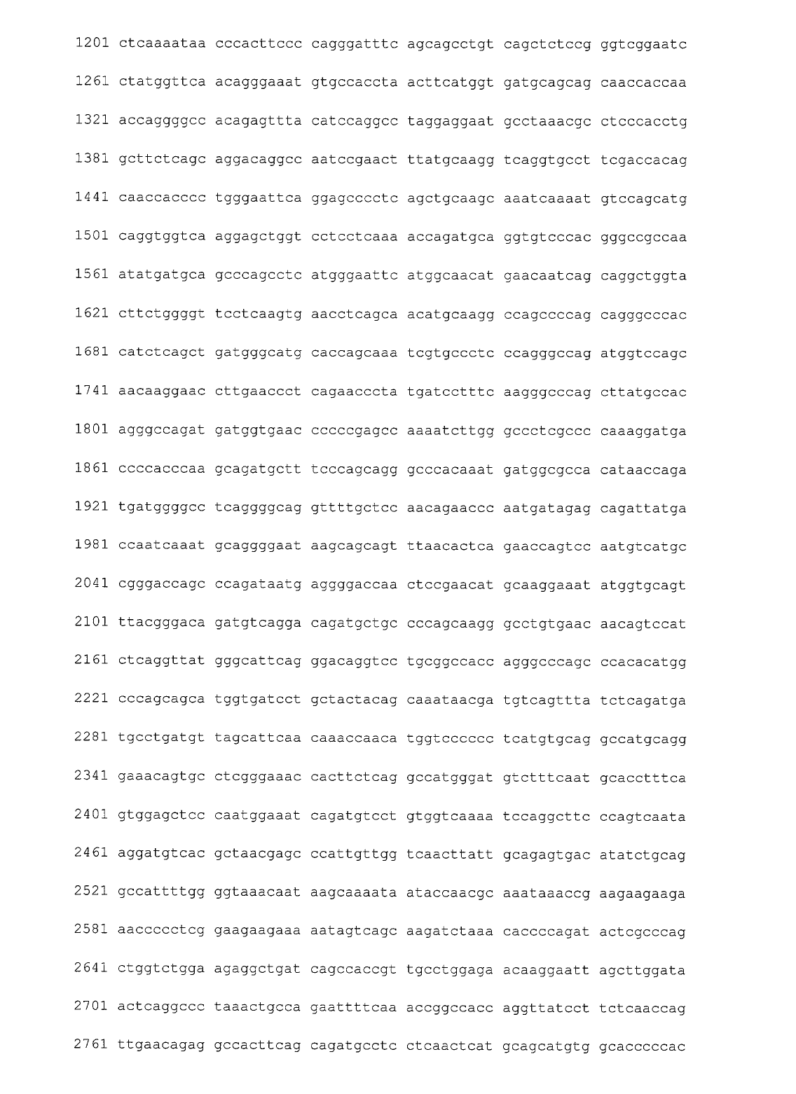

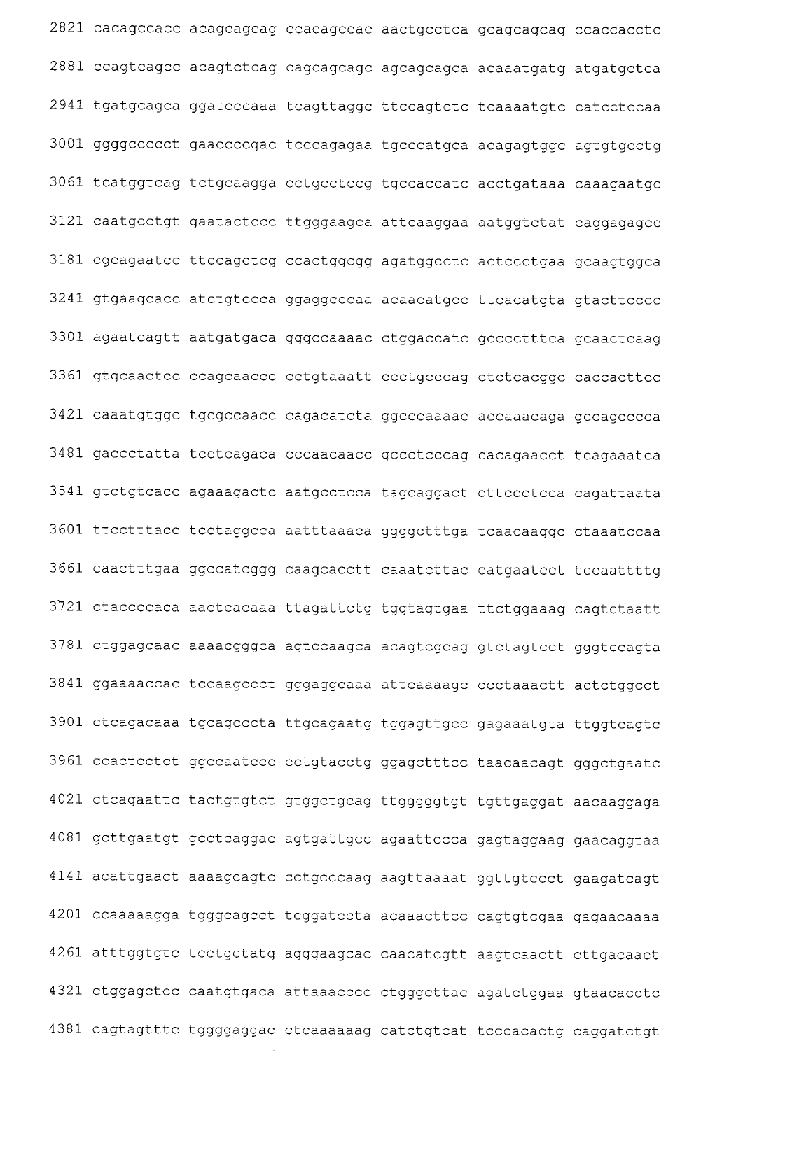

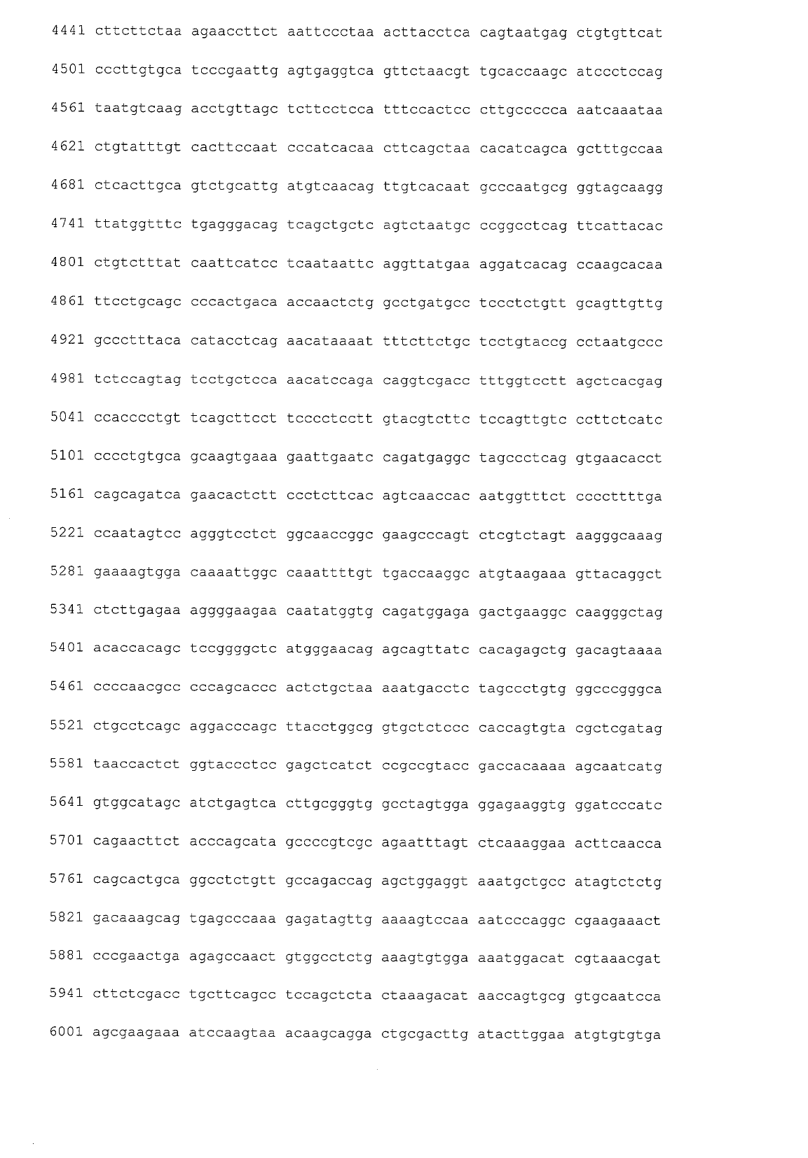

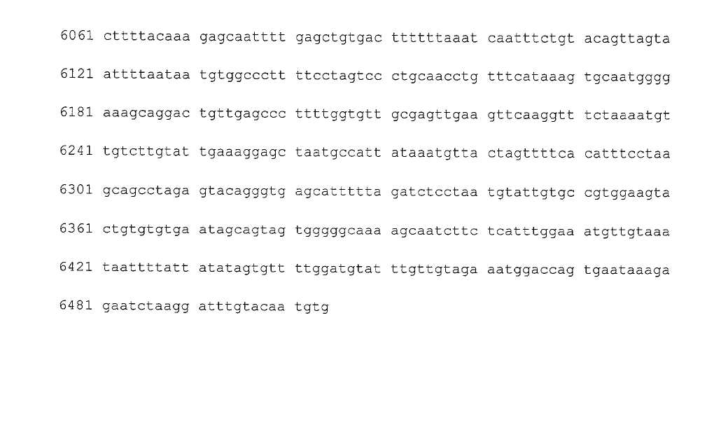

According to one aspect of the invention there is provided an isolated mammalian coactivator, RAP250, the coactivator comprising the amino acid sequence of FIG. 1A and polypeptides, other than the polypeptide of FIG. 11A (KIAA0181) comprising at least a portion of the amino acid sequence of FIG. 1A and having a biological activity of RAP250. Preferably, the coactivator consists of the amino acid sequence of FIG. 1A and comprises 2063 amino acid residues. A coactivator according to the invention may have less than 30% homology with known coactivators. The coactivator may have a molecular weight of about 250 kDa.

The invention also provides polypeptides having at least 70% sequence identity to the sequence of FIG. 1A. Preferably, the polypeptides have at least 90% sequence identity to the sequence of FIG. 1A or may have at least 90% sequence identity to the sequence of FIG. 1A. The invention also provides polypeptides, other than the polypeptide of FIG. 11A (KIAA0181) having at least 50% identity, preferably at least 60% identity, to amino acids 819-1096 of the sequence in FIG. 1A.

The biological activity of an isolated coactivator or polypeptide in accordance with the invention may be an interaction such as binding with a nuclear receptor such as at least one of TR, RXR, PPAR LXR or the ERs. A coactivator or a polypeptide according to the invention may bind to the nuclear receptor in a homo-or heterodimeric form. The nuclear receptor may be bound to DNA. An isolated coactivator or a polypeptide according to the invention may bind ERβ more strongly than ERα preferably at least about twice as strongly as it binds ERα.

An isolated coactivator or a polypeptide according to the invention may display ligand-dependent interaction with TR or ligand-enhanced interaction with ER, PPAR and RXR. Another biological activity of an isolated coactivator or a polypeptide according to the invention may be enhancement of transcription of at least one of TR, PPAR, LXR, ERα, ERβ, and RXR. A further biological activity of an isolated coactivator or a polypeptide according to the invention may be the ability to form a complex with a DNA-bound nuclear receptor such as TR, PPAR, RXR, LXR or ER.

The invention also provides a fusion protein comprising at least a portion of polypeptide according to the invention and a non-coactivator-related amino acid sequence.

An isolated coactivator or polypeptide according to the invention may have least one LXXLL amino acid motif, preferably two or more such motifs. The isolated coactivator or a polypeptide according to the invention may have a single functional NR box, preferably one including a single functional LXXLL amino acid motif. An isolated coactivator or a polypeptide according to the invention may have an N terminal Q-rich region flanked by two poly-Q stretches. An isolated mammalian coactivator according to the invention may be derived from human cells or mouse cells. An isolated mammalian coactivator according to the invention may be expressed in brain and reproductive organ cells. In humans it may be expressed at relatively high levels in at least one of reproductive organs such as ovary, testis or prostate; or peripheral blood leukocytes, brain and heart. It may be expressed in humans at relatively moderate levels in at least one of pancreas, kidney, liver, colon, spleen, and placenta, and at relatively low levels in at least one of small intestine, thymus, and skeletal muscle.

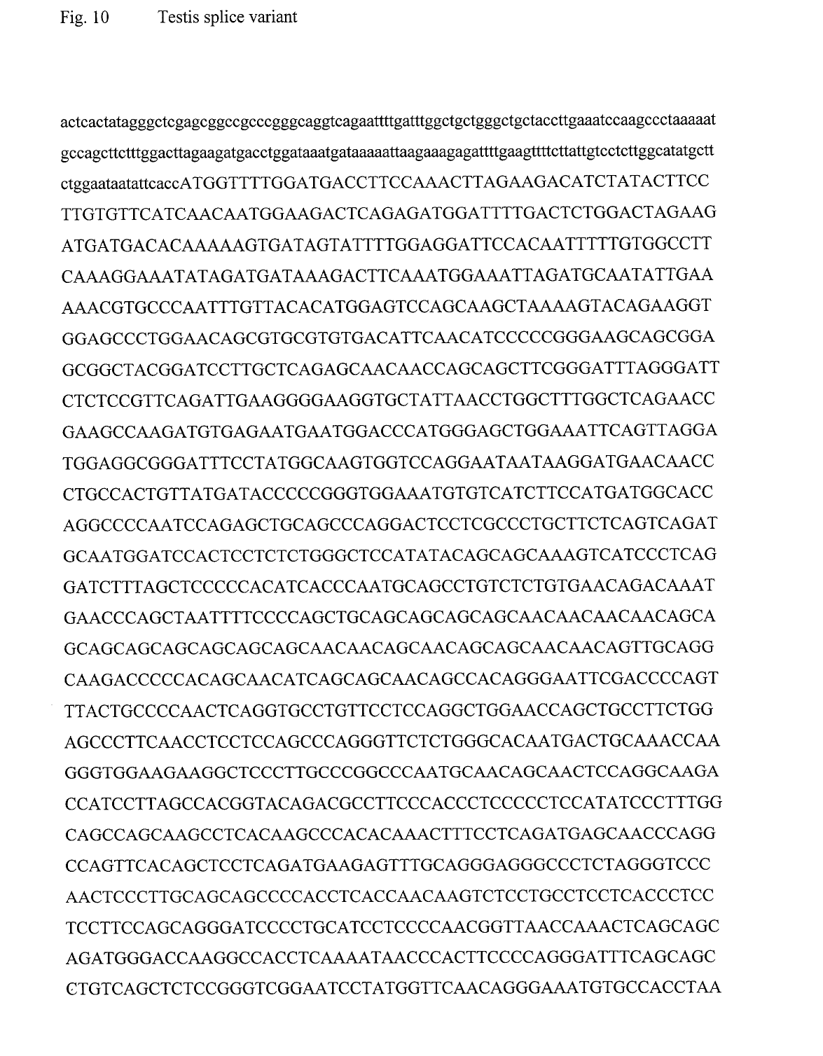

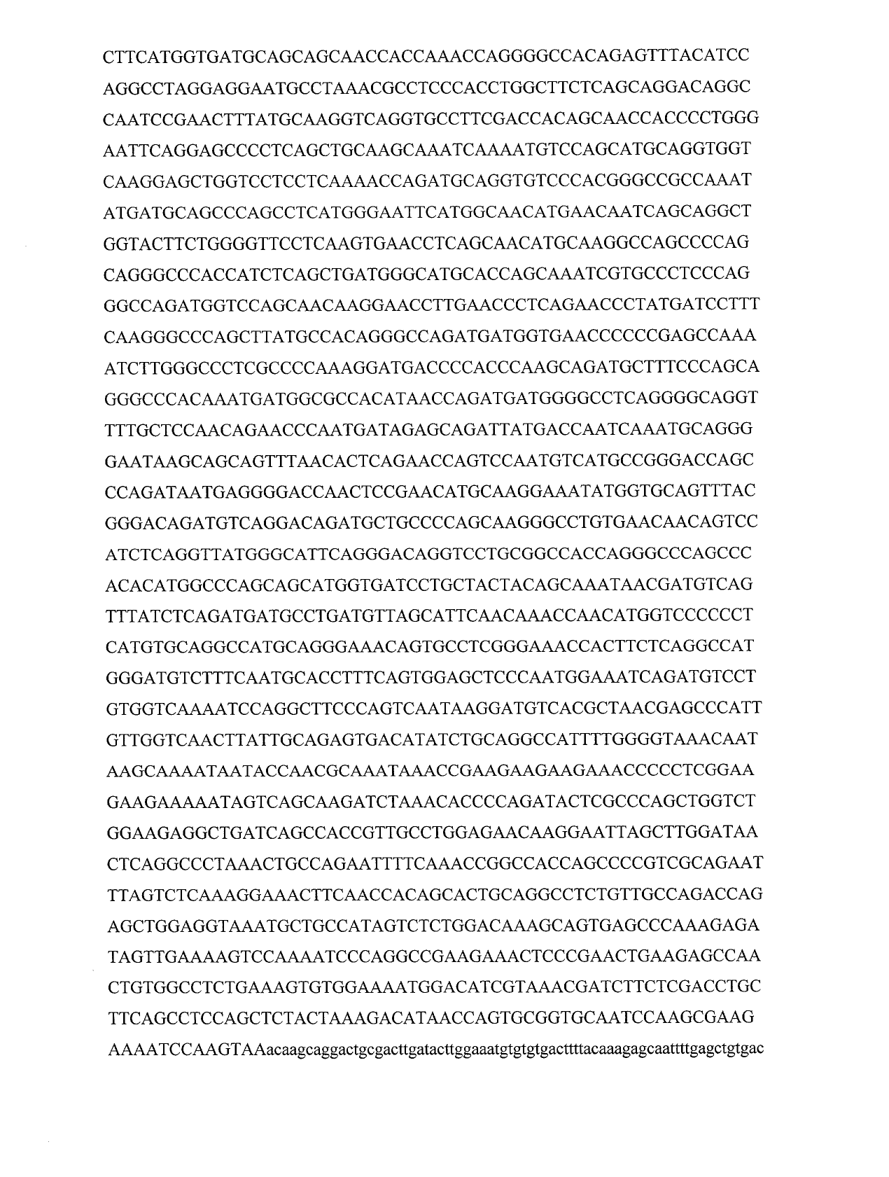

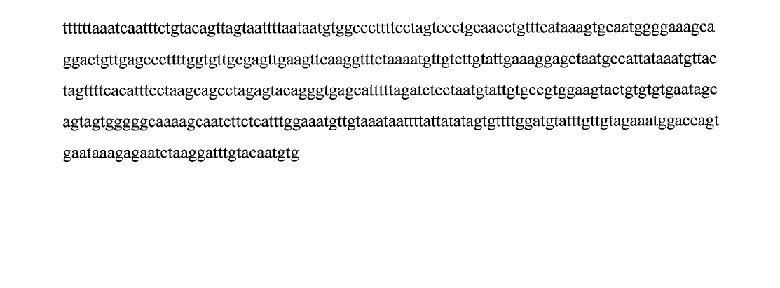

The invention also provides a nucleotide sequence encoding a mammalian coactivator or polypeptide according to the invention other than the nucleotide sequence of KIAA0181 (FIG. 11B). Preferably the nucleotide sequence comprises or consists of the DNA sequence of FIG. 1A or 10 or fragments thereof; DNA sequences which encode a polypeptide having the same amino acid sequences encoded by FIG. 1A or 10 or a fragment thereof, and sequences which hybridize thereto under stringent conditions.

As used herein, the term “hybridize [thereto] under stringent conditions” refers to the ability of a denatured DNA sequence to hydrogen bond to another denatured DNA sequence through complementary base pairing under conditions which allow sequences having at least 90% similarity to form such hybrids. Such conditions are well known in the art and are exemplified by salt and temperature conditions substantially equivalent to 5× SSC and 65° C. for both hybridization and wash.

The nucleotide sequence may comprise 6189 base pairs.

Besides the full-length cDNA sequences set forth herein, it will be readily apparent to those of skill in the art that any other DNA sequence which, as a result of degeneracy in the genetic code, encodes the same amino acid sequence as FIG. 1A or 10 is part of the applicant's invention. While those specific sequences are not set forth herein due to space considerations, it should be understood that one of ordinary skill in the art could ascertain all of such DNA sequences merely by reference to the genetic code and without the exercise of inventive skill.

In addition to genetically redundant DNA sequences, the invention also includes DNA sequences which encode other amino acid sequences which are at least 60%, and preferably at least 90% similar to FIG. 1A or 10. The identification and isolation of additional sequences may be achieved by standard DNA library screening techniques (hybridization, PCR) using the sequence of FIG. 1A or 10 or portions thereof as a probe. Such homologous sequences may be found in any mammalian tissue cDNA library, as well as in insect cDNA libraries, yeast and other fungi cDNA libraries and prokaryotic cDNA libraries.

According to an alternate embodiment, the invention provides isolated DNA sequences which encode RAP250-related polypeptides; DNA sequences which hybridize to either of the former DNA sequences; and DNA sequences which code for a polypeptide having the same amino acid sequence as any of the previous DNA sequences.

Additional DNA sequences which encode RAP250 related polypeptides may be identified by standard DNA library screening techniques using nucleotides of FIG. 1A or 10 or portions thereof as a probe. Even more preferred are homologous DNA sequences which contain nucleotides encoding only conservative amino acid substitutions for some or all of the other amino acids of FIG. 1A or 10. The translation products of any of these DNA sequences may then be used in assays or methods as described below.

As set forth above, DNA sequences according to this aspect of the invention may be identified and isolated using methods well known in the art, for example, through standard cDNA library screening. It will be appreciated by one of ordinary skill in the art that screening techniques such as those described in the art may be used to identify homologous genes in other species, and these DNA and amino acid sequences are also included in the present invention.

The invention also provides vectors including at least a portion of such a nucleotide sequence.

Assay systems can be used to screen for potential therapeutic compounds, including peptides and chemical ligands, which interact with the nuclear receptor coactivator. Such therapeutic compounds could act by either modulating the interaction of the nuclear receptor coactivator with an interacting protein or by modulation of the nuclear receptor coactivator directly. Interaction assays, include, but are not limited to, the two-hybrid assay and immunoprecipitation. Activity assays include, but are not limited to, mammalian transfection assays in which the RAP250 activation of a nuclear receptor response is measured.

The invention also provides a method of isolating nuclear receptor (NR) coactivators comprising detecting binding of putative NR coactivators with at least one nuclear receptor. For example the nuclear receptor may be selected from TR, ER, PPAR, and RXR. The binding may be performed in the presence of a nuclear receptor ligand.

The invention also provides probes for identifying coactivators comprising at least a portion of the sequence:

a. GCACCCCCACCACAGCCACCACAGCAGCAGCCACA
b. GCAAGGACCTGCCTCTGTGCCACCATCACCTG

Cells, tissues or organisms engineered to contain a nucleotide sequence or a vector according to the invention, or engineered to express a recombinant form of a coactivator or a polypeptide according to the invention are also contemplated.

The invention also provides a method of identifying nuclear receptor ligands, the method comprising testing the effect of a putative ligand on the interaction, particularly binding, of an isolated coactivator or a polypeptide according to any preceding claim and a nuclear receptor. The nuclear receptor may be an ER, TR, PPAR or RXR. The invention includes nuclear receptor ligands obtainable by such a method.

In particular the invention provides a method of identifying selective estrogen receptor modulators (SERMs), in which the effect of a putative SERM on the interaction, preferably binding, of an isolated coactivator or polypeptide according to the first aspect of the invention with ERα and ERβ is tested. The invention includes ER ligands obtainable by such a method.

The invention also provides a pharmaceutical composition, comprising an isolated coactivator or a polypeptide according to the first aspect of the invention or a ligand or compound obtained or identified by a method according to the invention, and methods of treating a nuclear receptor-related disorder or condition in a subject comprising supplying the subject with such a pharmaceutical composition.

Pharmaceutical compositions of this invention comprise any of the compounds of the present invention, and pharmaceutically acceptable salts thereof, with any pharmaceutically acceptable carrier, adjuvant or vehicle. Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of this invention may be administered orally, parenterally, by inhalation spray, topically, rectally, nasally, buccally, vaginally or via an implanted reservoir. We prefer oral administration or administration by injection. The pharmaceutical compositions of this invention may contain any conventional non-toxic pharmaceutically-acceptable carriers, adjuvants or vehicles. The term parenteral as used herein includes subcutaneous, intracutaneous, intravenous, intramuscular, intra-articular, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono-or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical compositions of this invention may be orally administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Estrogen receptor-related disorders that may be so treated include cancer of the breast and uterus; endometriosis; cardiovascular conditions; hot flushes; psychological conditions such as depression, mood etc; anti-inflammatory indications such as asthma, upper airway; and osteoporosis. In particular the invention provides a method of restoring wildtype RAP250 function in a subject, the method comprising supplying the subject with a nucleotide sequence or a vector according to the invention.

According to another aspect of the invention there is provided a coactivator having an amino acid sequence derived from the nucleotide sequence of FIG. 10 or a portion of that sequence. Preferably the coactivator is expressed only in testis.

According to another aspect of the invention there is provided an isolated mammalian coactivator of about 6 kilobase pairs in size and which is expressed only in peripheral blood leukocytes.

The results of in vitro and in vivo experiments indicate that the interaction of RAP250 with nuclear receptors is ligand-induced and involves only one short LxxLL motif called an “NR-box”. Transient transfection assays further demonstrate that RAP250 has a large intrinsic glutamine-rich activation domain and can significantly enhance the transcriptional activity of several nuclear receptors with different affinities. In particular, our data provide evidence that RAP250 functions as a preferential coactivator for estrogen receptor β (ERβ)-mediated transactivation. Interestingly, Northern blot and in situ hybridization analyses reveal that RAP250 is expressed in a tissue-specific manner with the highest expression in reproductive organs (testis, prostate and ovary) and brain, organs known to express high levels of estrogen receptors. Together, our data suggest that RAP250 may play an important role in the ER signalling pathway in human reproductive organs by modulation of ERβ-mediated transactivation.

Definitions

The term “coactivator” as used herein means a protein or polypeptide which directly or indirectly interacts in vitro or in vivo with a nuclear hormone receptor resulting in an increased nuclear hormone activity as defined by a transcription assay for example (or the assays defined herein). The interaction may be nuclear hormone dependent or independent and may be transient. Preferably, the protein or polypeptide shares at least 90% identity with the amino acid sequence of FIG. 1A.

The term “PPAR” as used herein embraces at least PPARα and PPARγ. Coactivators in accordance with the invention will now be described, by way of example only, with reference to the accompanying drawings, FIGS. 1 to 12 in which:

FIG. 1 Full length cDNA of human RAP250 (A) Complete deduced amino acid sequence from human RAP250 cDNA. (B) In vitro translation and visualisation of RAP250 cDNA. (C) Alignment of mouse RAP250 partial clone and the homologous region on human protein. (D) Complete deduced amino acid sequence from human RAP250 cDNA.

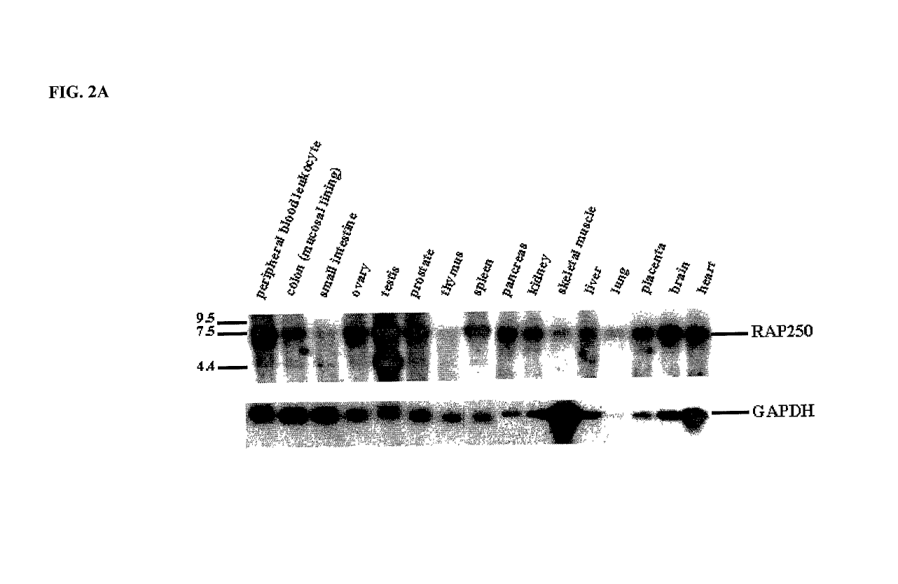

FIG. 2 (A) Comprises a Northern blot tissue distribution of RAP250 mRNA. (B) RAP250 mRNA expression in mouse embryo (Ba-Bc), postnatal and adult mouse tissues (Bd) and adult rat tissues (Be-Bh).

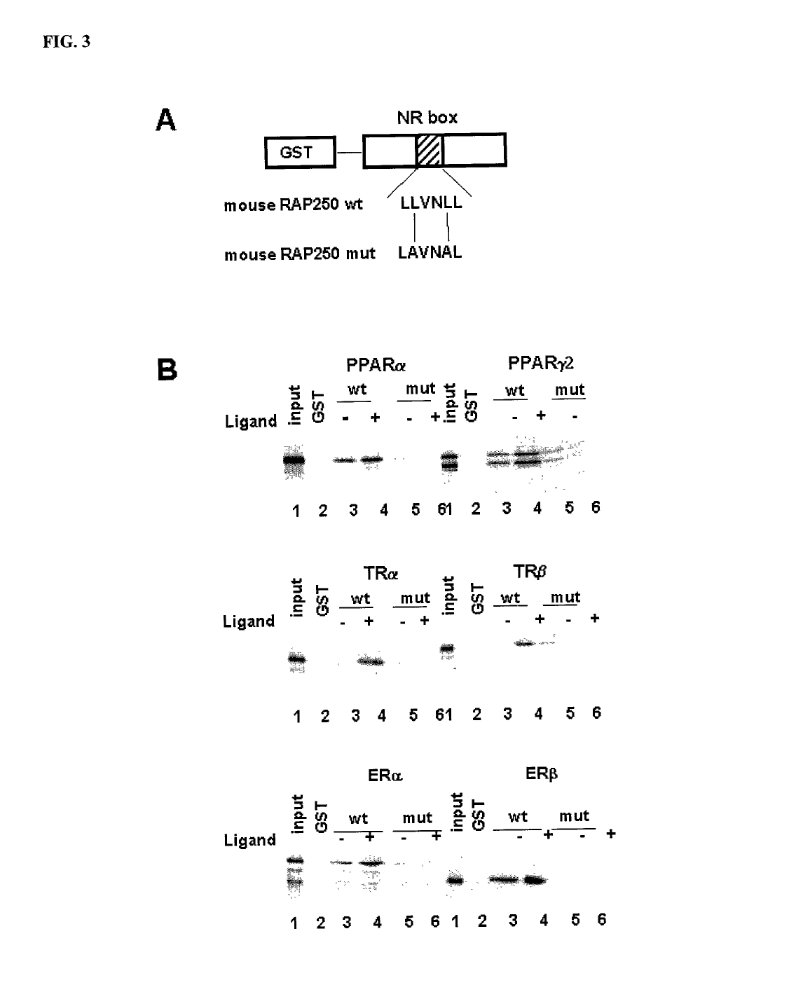

FIG. 3 comprises schematic representations and gels which illustrate in vitro interaction between mRAP250 and TRs, PPARs and ERs. (A) Is a schematic representation of GST protein fused to wild type (wt) and mutated (mut) N-R box containing fragments of mRAP250 used in a pull down assay with in vitro translated NRs. (B) Addition of ligand enhances RAP250 interaction with PPARs, TRs and ERs.

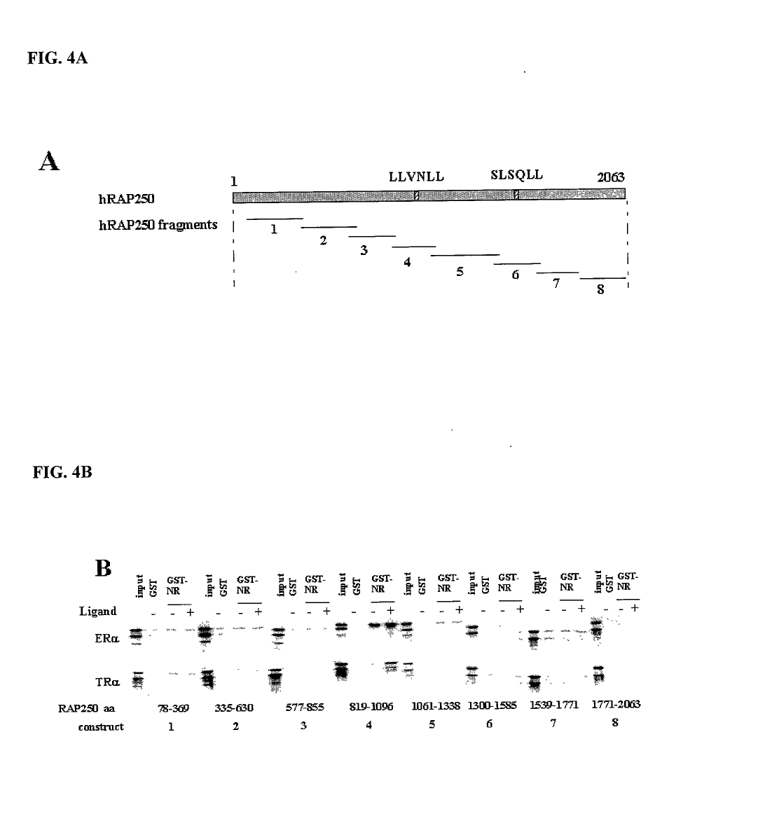

FIG. 4 comprises a schematic representation and gels which show the NR interaction domain of RAP250 contains only one functional N-R box. (A) Linear diagram of the RAP250 deletion constructs used in this study. (B) Analysis of interaction between 8 deletion constructs of RAP250 and GST-fused nuclear receptors.

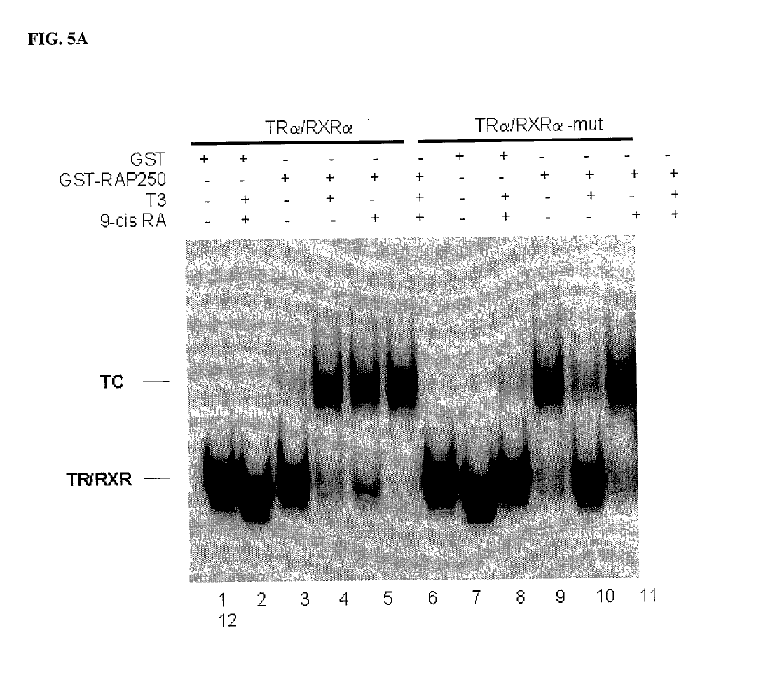

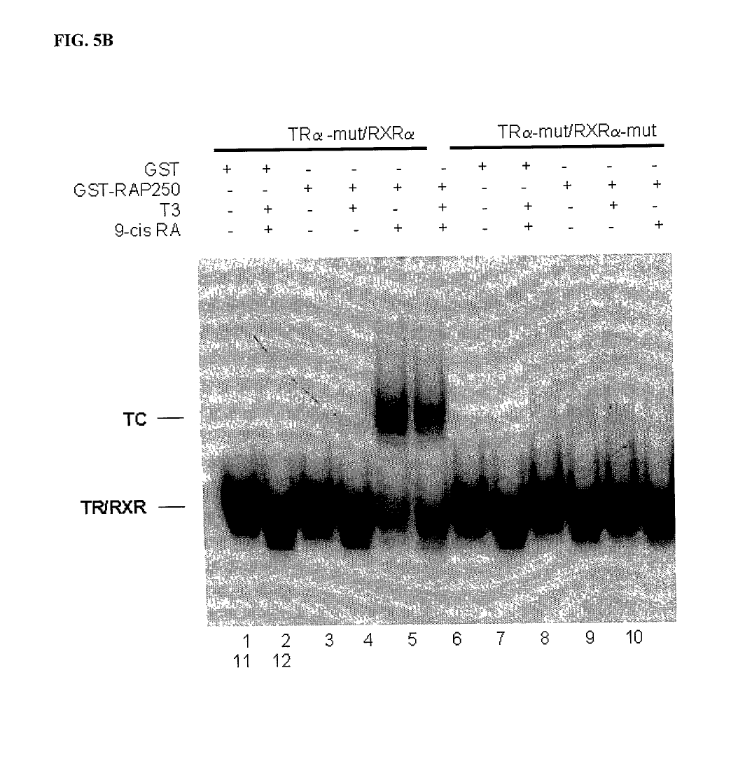

FIG. 5 comprises schematic representations and gels which show that RAP250 forms an oligomeric complex with DNA bound NRs. EMSA analysis with in vitro translated NRs and bacterially expressed GST-RAP250 (a. a. 818-931) protein. (A) RAP250/TRα/RXRα oligomeric complex formation on a TRE (Direct Repeat+4). (B) RAP250/TRβ/RXRα oligomeric complex formation on a TRE (Direct Repeat+4).

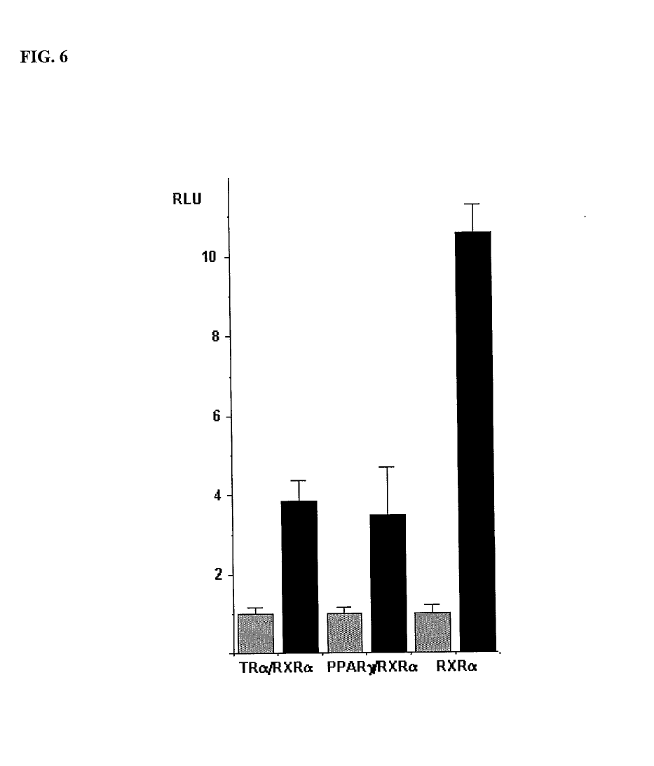

FIG. 6 comprises a graph which shows that RAP250 strongly interacts with nuclear receptors in mammalian cells.

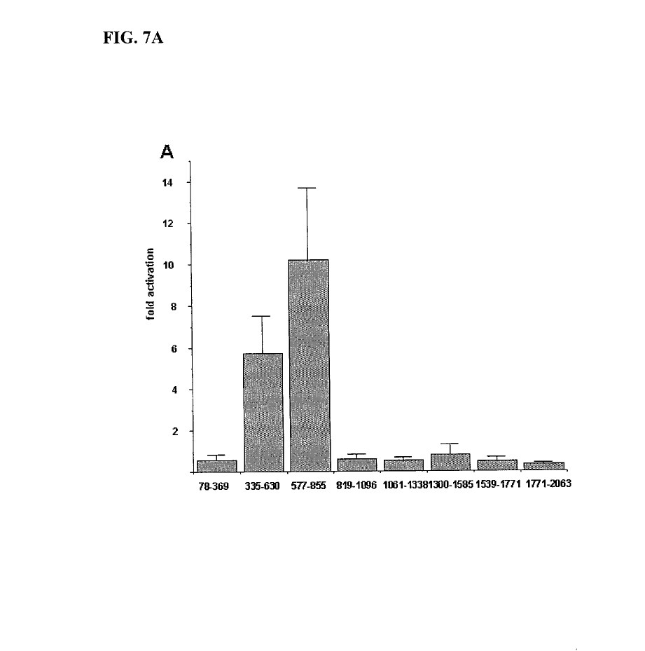

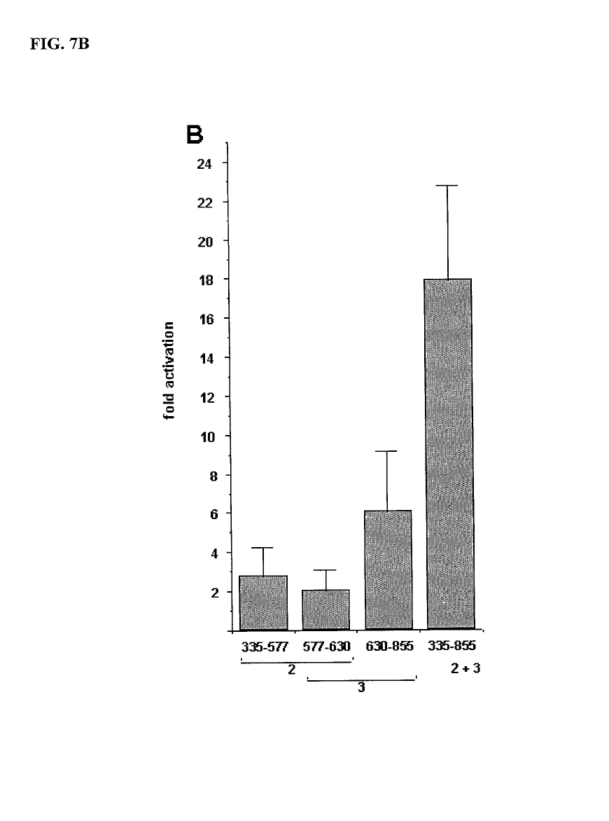

FIG. 7 comprises graphs which illustrate localisation of a transcription activation domain in RAP250.

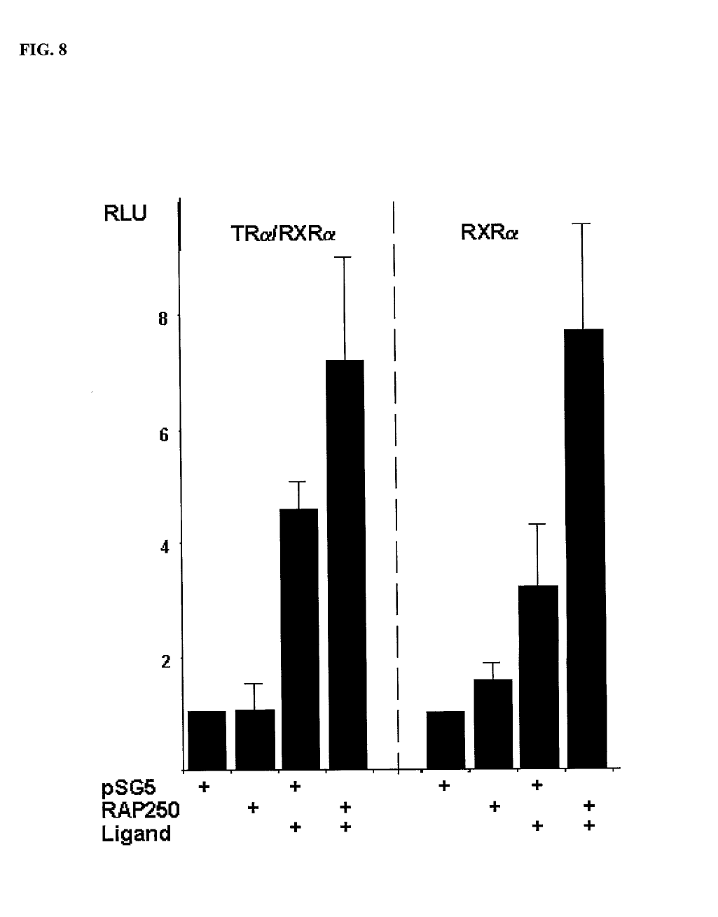

FIG. 8 comprises graphs which illustrate that human RAP250 enhances the transcriptional activities of wild type TRα and RXRα. Cos 7 cells were transiently co-transfected with 0.5 μg of luciferase reporter plasmid (containing DR₄ for TRα, DR₁ for RXRα), 0.5 μg of each reporter construct (TRα and RXRα) and 0.5 μg of pSG5-RAP250 or 0.5 μg of pSG5, in absence or presence of the appropriate ligand (20 nM T₃, or 10 μM 9-cis retinoic acid). The activity of the luciferase reporter gene was measured after 24 h after addition of ligands. Results represent mean standard ± standard deviation of at least two separate experiments carried out in duplicate, and were normalised to the activity of each NR/reporter combination transfected with pSG5 without ligand, which was set as 1.

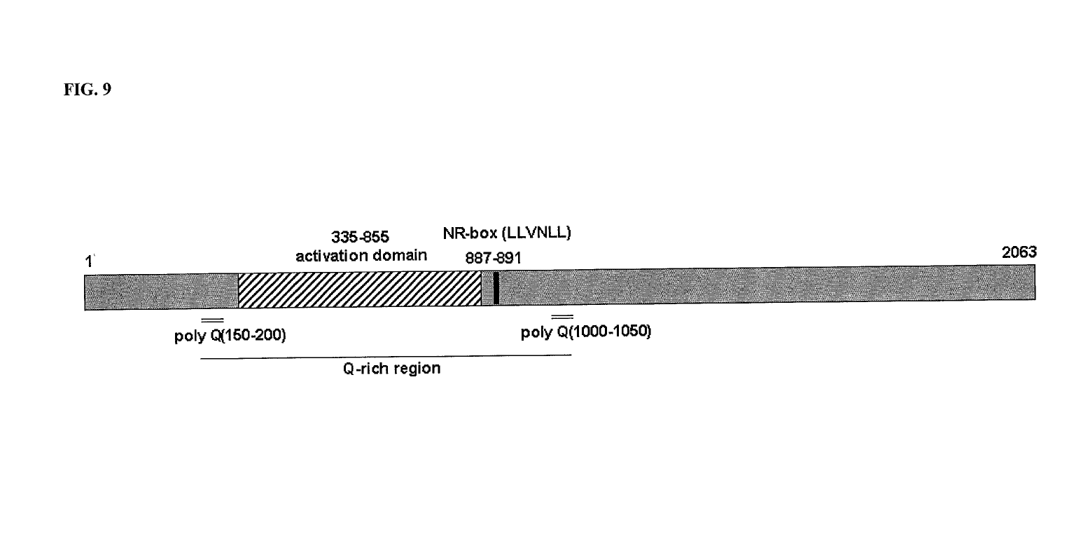

FIG. 9 is a schematic representation of RAP250.

FIG. 10 is the cDNA sequence of a testis splice variant of hRAP250.

FIG. 11 shows a) the predicted amino acid sequences and b) the mRNA sequence of KIAA0181.

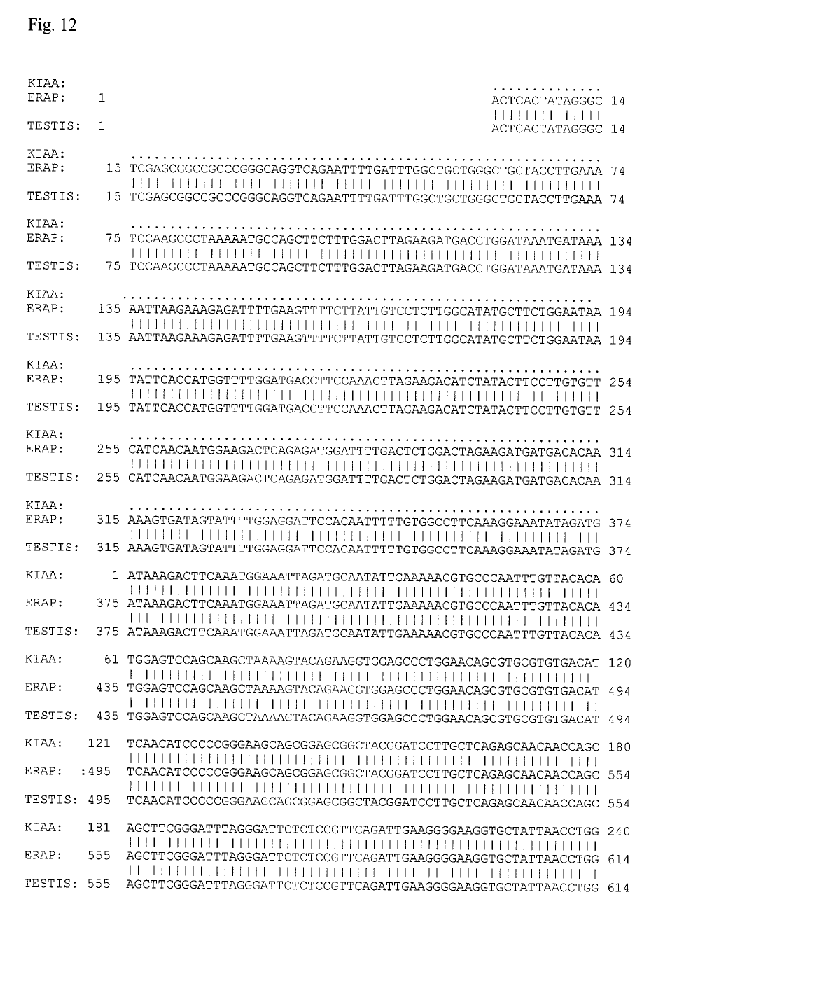

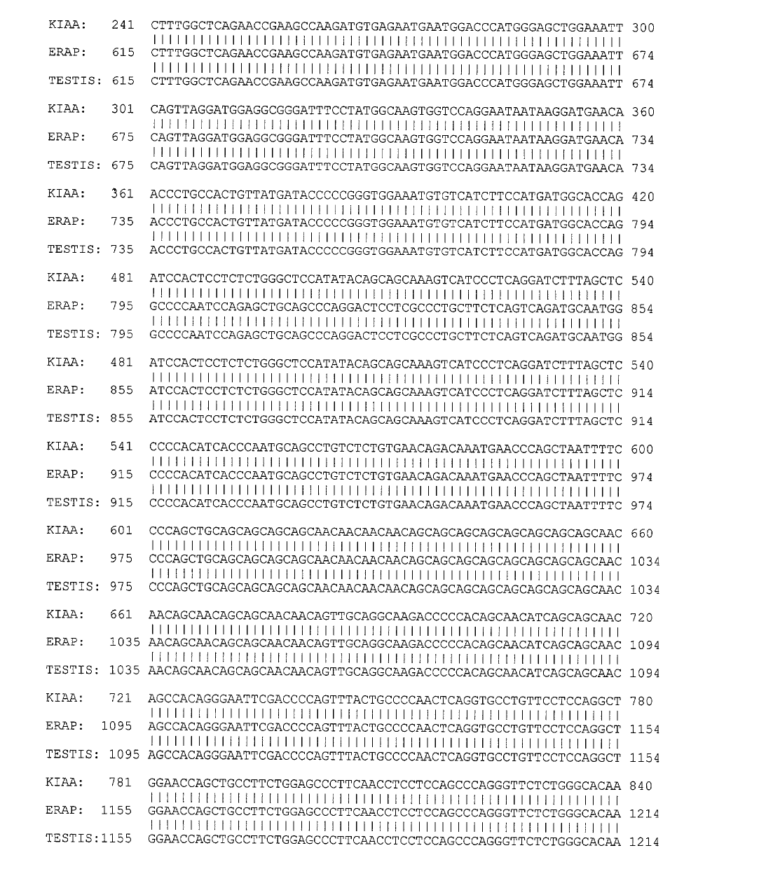

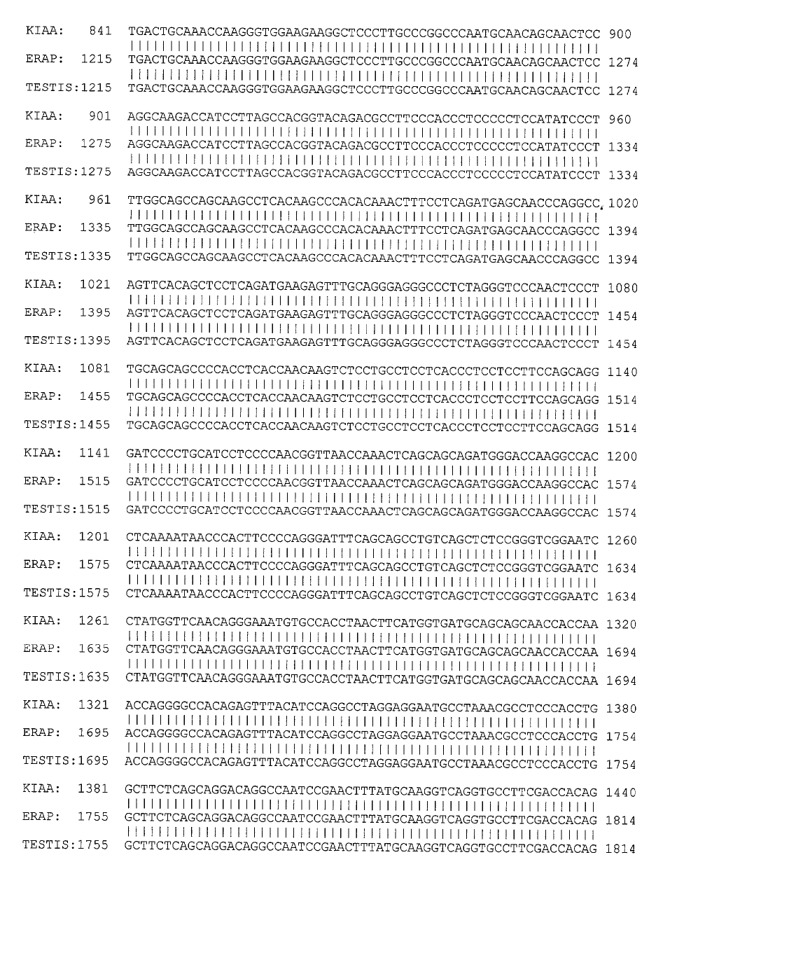

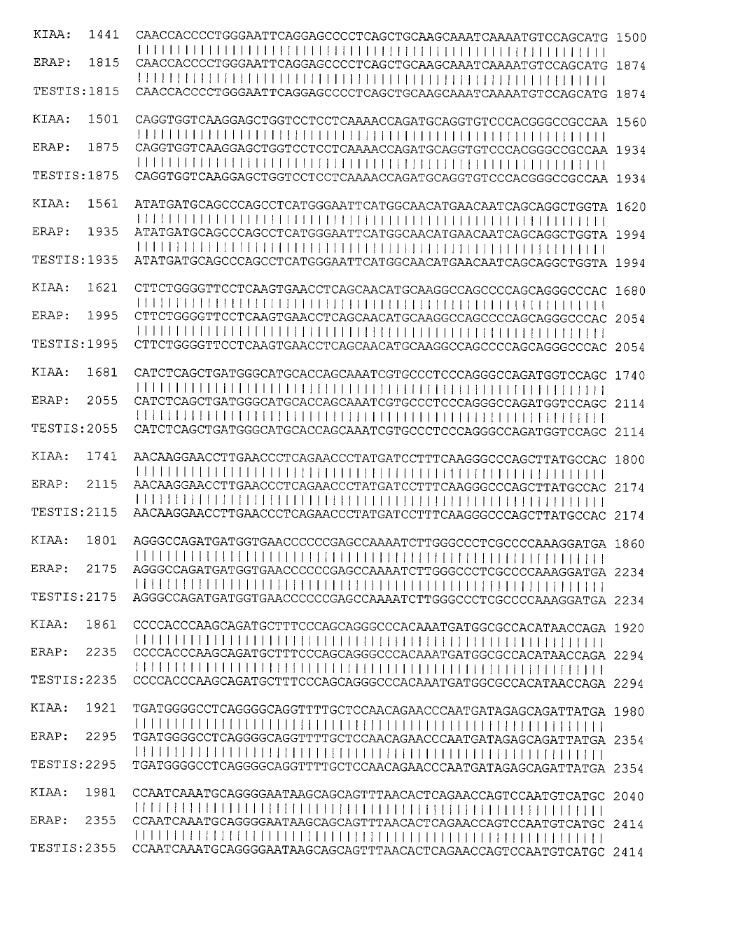

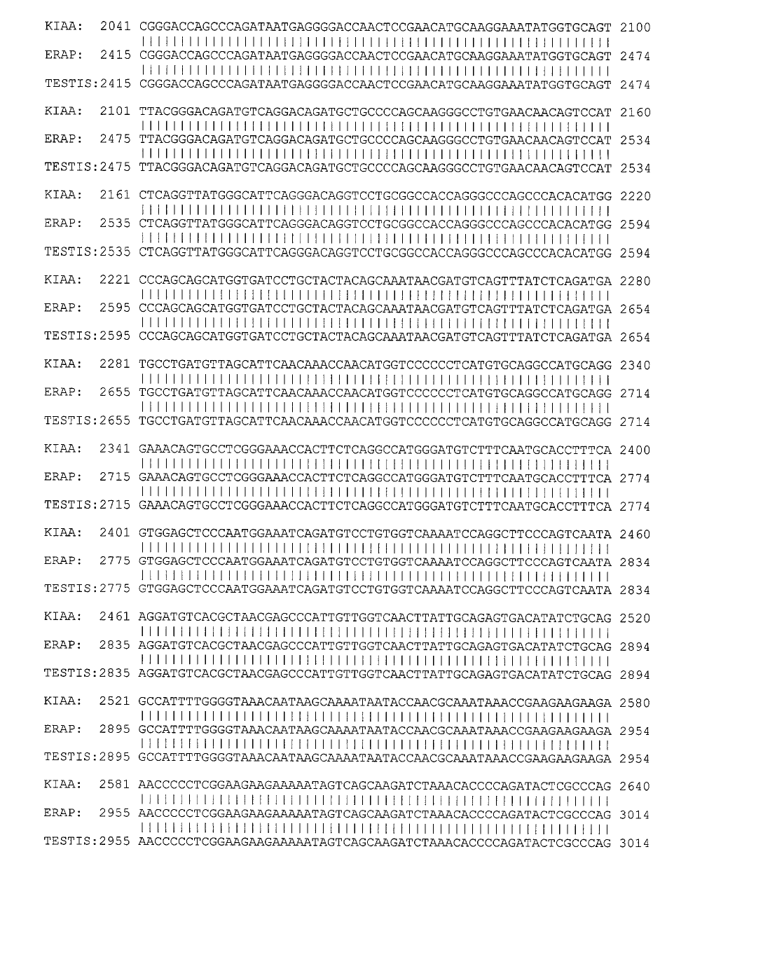

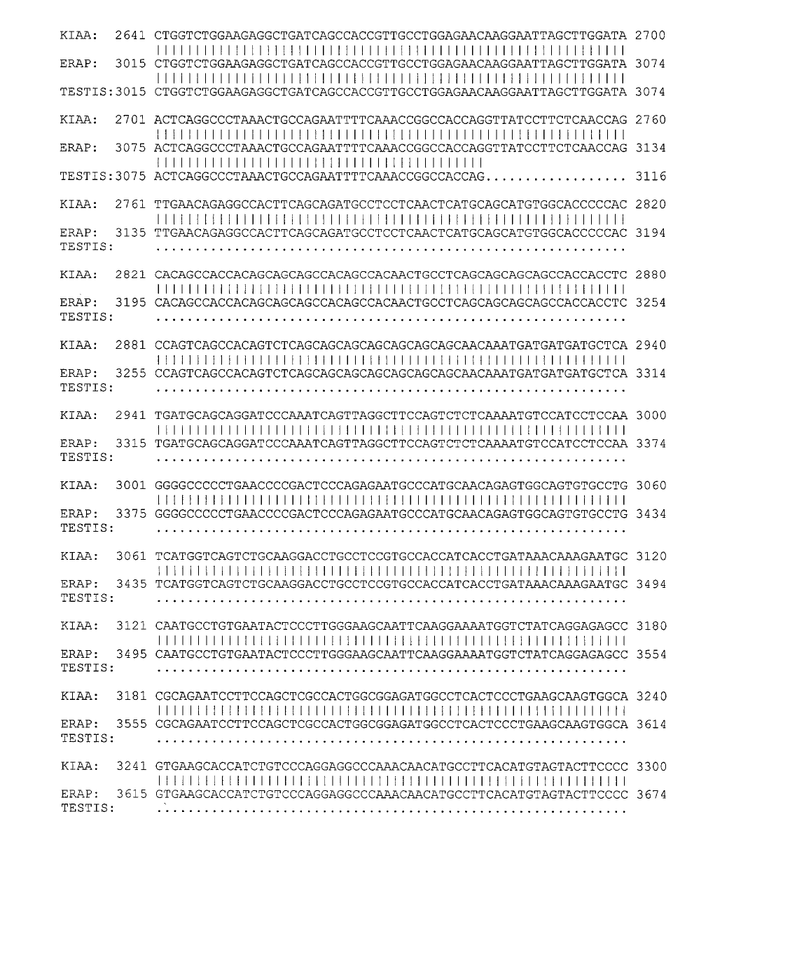

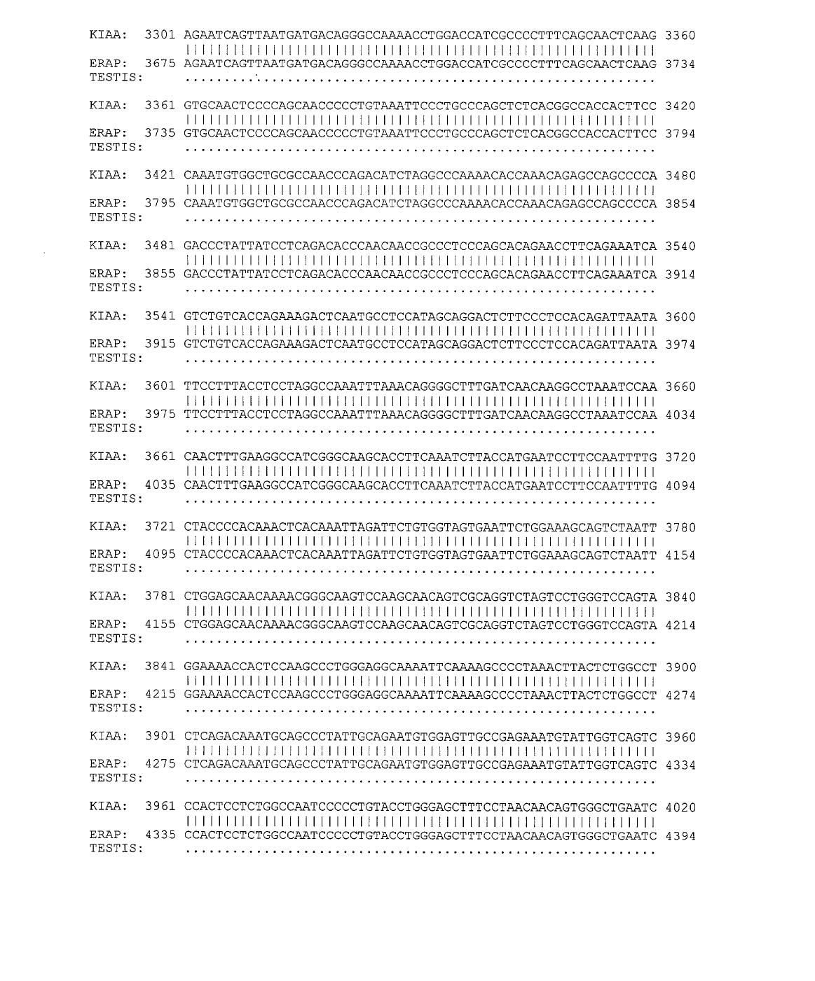

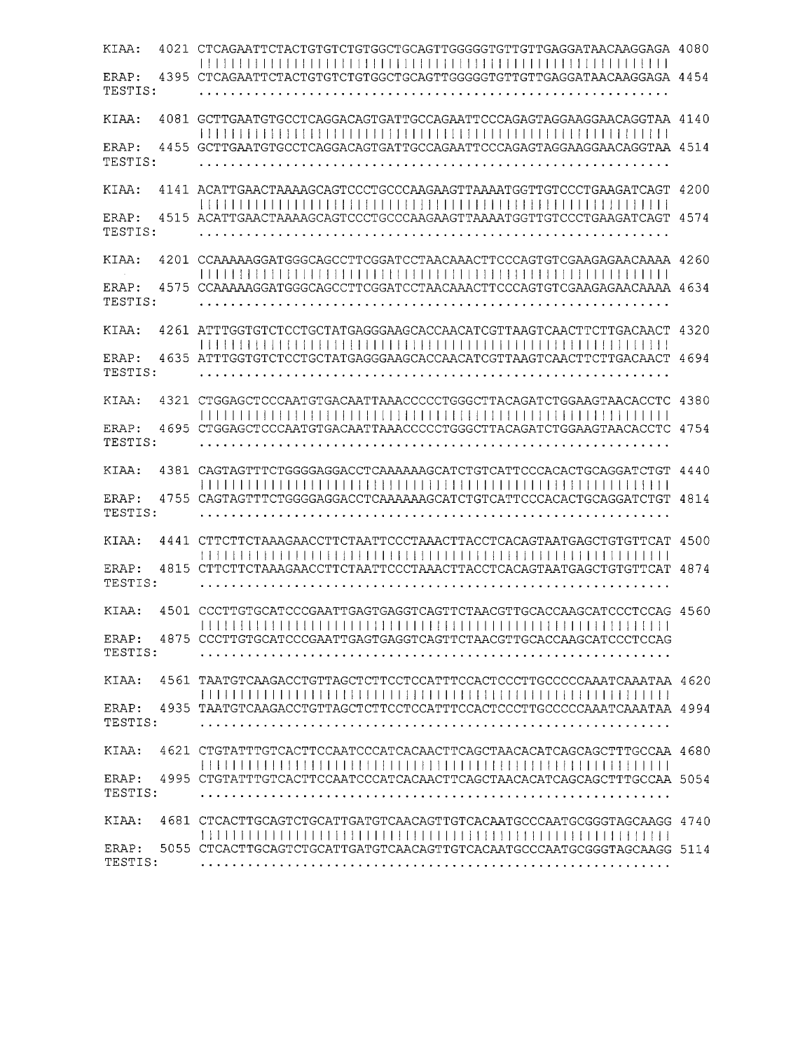

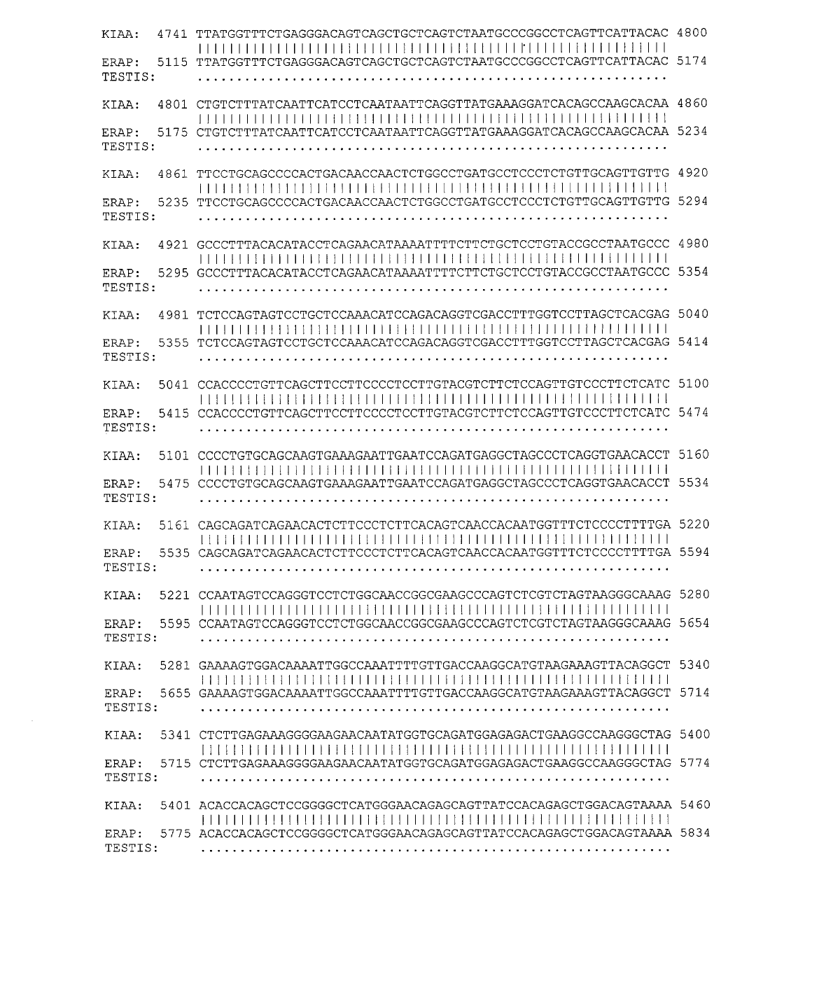

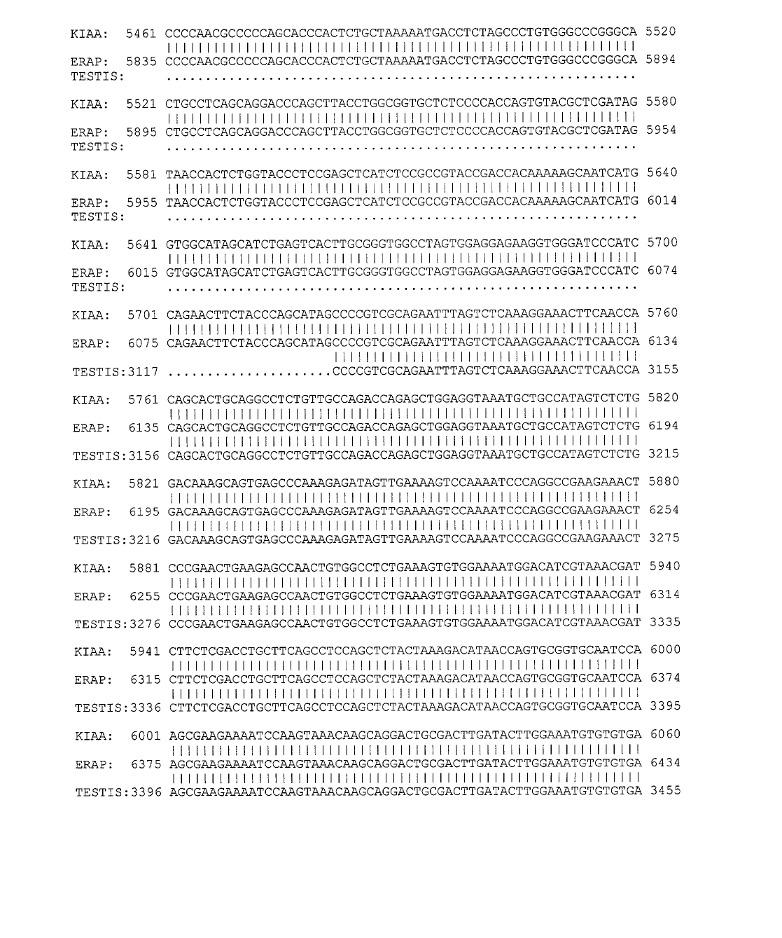

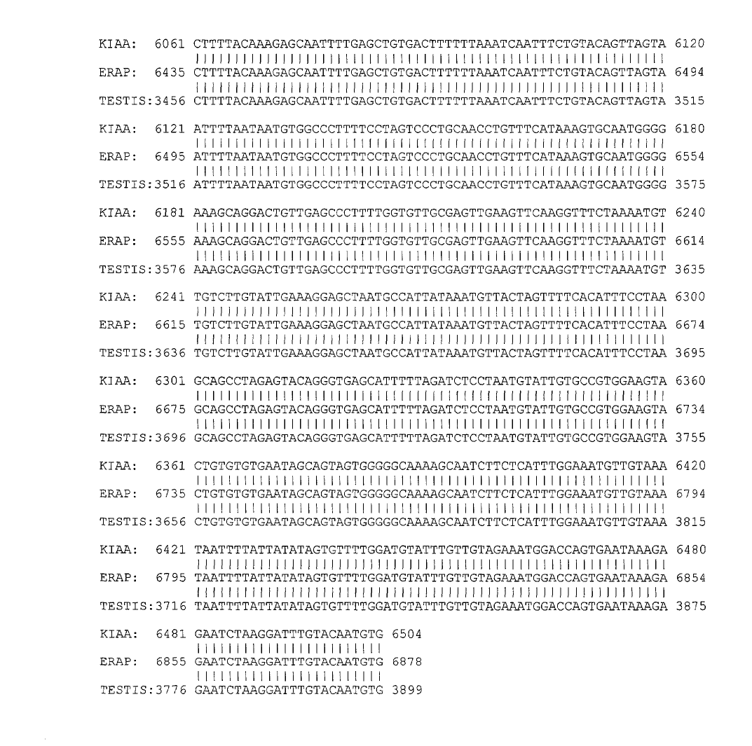

FIG. 12 is an alignment of the nucleotide sequences of KIAA0181 and RAP250 and its testis variant.

1) Plasmids

All constructs were generated using standard cloning procedures and verified by restriction enzyme analysis and DNA-sequencing. The partial mouse RAP250 cDNA fragment encoding amino acids 782-1138 was released from the EcoRI site of the pGAD10 clone isolated by the yeast two-hybrid system (Clontech) and subcloned into the EcoRI site of pGEX-4T1 vector (Amersham Pharmacia Biotech). GST-hERα (aa 249 to 595) and GST-hTRα (aa 122 to 410) have been described previously (Johansson, L., et al, supra; Leers, J., et al (1998) Mol. Cell Biol. 18: 6001-6013). Mutated variant of RAP250 was constructed by two independent PCRs with the flanking primers and two mutagenesis primers:

5′-TTAACGAGCCCATTGGCGGTCAACGCACTACAGAGTGAC-3′ and
5′-GTCACTCTGTAGTGCGTTGACCGCCAATGGGCTCGTTAA-3′

The corresponding PCR products were isolated and combined together by an additional PCR with the flanking primers. The product of this PCR was isolated by agarose gel, digested by EcoRI and cloned into the corresponding site of pGEX-4T1. GST-RAP250 (aa 818 to 931) was generated by PCR and cloned into the EcoRI/SalI sites of pGEX-4T1. Nuclear receptors for in vitro translation have been expressed from the following previously described plasmids: pBKCMVrPPARα (T3), pSG5mPPARγ2 (T7), pCMVhTRα (T3), pCMVhTRβ (T7) (Treuter, E., et al, supra), pBKCMVrRXRa (T3) (Wiebel, F. F., and Gustafsson, J. -Å (1997) Mol. Cell. Biol. 17: 3977-3986), pT7hERα (aa 1-595) and pT3hERβ (aa 1-485) (Johansson, L., et al (1999) J. Biol. Chem 274: 345-353).

Plasmids with mutated forms of TRα and RXRα have been described previously (Wiebel, F. F., et al, supra; Saatcioglu, F., et al (1993) Mol Cell. Biol, 13: 3675-3685). RAP250 deletion constructs were made by PCR and subcloned into the NotI/NheI sites of the plasmid pSG5Gal4(GBT9J) (Stratagen). These plasmids were used both for transient transfection experiments and/or to in vitro translate RAP250 partial fragments. The luciferase reporter gene, the Gal4 reporter construct UAS-tk-Luc has been described previously (Johansson, L., et al, supra). The following eukaryotic expression vectors were used to express NRs: pSG5mPPARγ2, pSG5-cTRα. (Saatcioglu, F., et al, supra) and pCMX-rRXRα (Wiebel, F. F., et al, supra). pVP16-mRAP250 (aa 782-1138) was made by subcloning an EcoRI fragment from pGAD10-mRAP250 (a clone orginally isolated from mouse) into pCMV-VP16. The reporter plasmids used were: DR4-tk-Luc (Wiebel, F. F., et al, supra), PPRE-tk-Luc (Treuter, E., et al, supra) and DR1-tk-Luc (Feltkamp, D., et al, (1999) J. Biol. Chem. 274: 10421-10429). pSG5-RAP250 plasmid used to transfect mammalian cells in the coactivation assay was obtained by subcloning the human full-length RAP250 cDNA (obtained by fusion of the 5′ end cDNA to the KIAA0181 plasmid) into pSG5 vector.

2) Yeast Two-Hybrid Screening and 5′-RACE PCR

To isolate cDNAs encoding proteins that interact with PPARα, yeast two-hybrid screening was carried out as described previously for the isolation of hRIP140 (Treuter, E., et al, supra). As bait, Gal4-PPARα LBD/AF-2 was used to screen a mouse embryo cDNA library (CLONTECH) in the vector pGAD10. One clone revealed no homology with any characterized proteins, but a strong homology with a human EST sequence of 6504 bp named KIAA0181 (Nagase, T., et al (1996) DNA Res. 3: 17-24). The Kazusa DNA Research Institute provided us with the human homologue cDNA. We used RACE-PCR to obtain the remaining 5′ end sequence of the human RAP250. This PCR amplification was performed using human testis marathon ready cDNA (CLONTECH) as template. The first amplification was performed using the adaptor primer 1 from the kit and the gene-specific primer (5′-ATAGGAAATCCCGCCTCCATCCTA-3′) for 30 cycles followed by a final elongation of 7 min. Each cycle consisted of 10 s at 94°, 10 s at 63° and 1 min 30 s at 68°; 1 ml of the PCR product was used as a template for the second amplification with the adaptor primer 2 from the kit and the nested gene-specific primer (5′-CTGGTTGTTGCTCTGAGCAAGGAT-3′) for 30 cycles, essentially using the same conditions as those used for the first amplification. The PCR product was cloned into pGEM-T (Promega) and 10 independent clones were sequenced.

3) Isolation and Cloning of RAP250 cDNA

We used the yeast two-hybrid system to screen a mouse embryo cDNA library with PPARα-LBD as a bait as previously described (Treuter, E., et al, supra; Leers, J., et al, supra). Of the isolated clones, more than 50% were isoforms of RXR, and a majority of the other clones were interacting parts of SMRT, N-CoR, TIF-2 and TRAP220 as described by Treuter et al. (Treuter, E., et al, supra). However, one of the interacting clones revealed no homology with any described protein and database searches revealed a strong homology with a human EST sequence of 6504 bp named KIAA0181 (Nagase, T., et al, supra). Compared to the human clone (FIG. 1A), this positive mouse clone only contained a partial cDNA sequence of 1.1 kb encoding 355 amino acids, corresponding to amino acids 782 to 1138 of the human sequence (FIG. 1A) and showing 90% identity with the human protein (FIG. 1C). The human clone contained a long open reading frame starting at nucleotide position 60 and a stop codon at nucleotide 6020. A downstream polyadenylation signal was also present in this cDNA indicating that the 3′-end of the gene was present. However, the first in-frame ATG codon of this human clone was not in an optimal context for translational initiation (no perfect Kozak site (Kozak, M. (1989) J. Cell. Biol. 108, 229-241), no in-frame stop codon upstream of this first methionine), suggesting that it perhaps was not the real initiation codon. In order to get a longer 5′-end sequence, we performed a 5′-RACE PCR and amplified an additional sequence of 433 nucleotides, among which were 231 nucleotides encoding 77 additional amino acids. The nucleotide sequence of the reconstituted full-length cDNA is 6878 bp in length, it contains a short 5′-untranslated region (UTR) of 202 bp with an upstream stop codon in frame with the first methionine, a longer 3 ′-UTR of 484 bp and a 6189 bp open reading frame which encodes a protein of 2063 amino acids with a calculated size of 250 kDa (FIG. 1A). The beginning of the coding sequence was defined by the first ATG downstream of an in-frame stop codon at position −51. The sequence (ACCATGGTTTTG) surrounding ATG essentially conforms to the Kozak site (A/Gcc ATG Gat) (Kozak, M. supra). To determine the size of the protein we performed an in vitro translation. As shown in FIG. 1B, the size of the in vitro synthesized protein is 250 kDa, which is consistent with the calculated size. This protein was designated RAP250 (nuclear Receptor Activating Protein 250) to signify both its coactivation function as described below and its size in kilodaltons. The human RAP250 shows some specific features such as a Q-rich region flanked by two poly-Q stretches in the N-terminal region (see FIG. 1A) and two copies of the LxxLL motif (LVNLL aa 887-891 and LSQLL, aa 1494-1495). Out of the two LxxLL motifs present in the human sequence, the mouse partial clone only contains the first one, which is identical in both species (FIG. 1C).

FIG. 1 RAP250 sequence. A, Complete deduced amino acid sequence of human RAP250 cDNA. The longest open reading frame of hRAP250 starts with the first methionine. The longest ORF of KIAA0181 cDNA clone obtained from Kazusa DNA Research Institute (Nagase, T., et al, supra) starts at methionine +78, while the cDNA sequence encoding the first 77 amino acids was obtained by RACE-PCR. RAP250 contains two LxxLL motifs (boxed residues 887-891 and 1491-1495). The boxed part on protein sequence represents the location on human sequence of the partial clone originally isolated from the mouse embryo cDNA library (aa 782-1138). RAP250 contains two poly-glutamine stretches (underlined) and in between, a region rich in glutamine (20% Q). The complete human protein sequence contains 2063 residues with a predicted molecular mass of 250 kDa. The nucleotide sequence contains an in-frame stop codon upstream of the first methionine (data not shown).

B, In vitro translation of RAP250 cDNA. The human full-length RAP250 cDNA cloned in pSG5 was in vitro transcribed and translated using rabbit reticulocyte lysate with [35S] methionine. Radiolabelled protein was fractionated on a 7% SDS-PAGE and visualized using autoradiography. The size of hRAP250 was estimated to 250 kDa by comparison to the size of mPBP (TRAP220) (Zhu, Y. J., et al (1997) J. Biol. Chem. 272: 25500-25506) and the molecular markers.

C, Alignment of the mouse RAP250 partial clone and the homologue region on human protein. White boxed residues in mouse RAP250 (bottom line) differs from the human corresponding residues (black boxed upper line). The two RAP250 sequences share 90% identity. The 55 nucleotides surrounding the LxxLL motif (LVNLL) are identical in both species, providing a large consensus sequence within the interacting region.

4) Northern blot

Human multiple tissue Northern blots with approximately 2 mg poly(A)+RNA per lane were hybridized according to the protocol of the manufacturer (CLONTECH). The probe used to detect RAP250 expression was a 0.8 kb EcoRI fragment (nucleotides 1104-1828) of hRAP250 cDNA radioactively labeled by the random-prime method (Rediprime, Amersham). GAPDH cDNA was also used as a control probe. Some variations of the control GAPDH levels were observed, showing a strong expression in skeletal muscle and heart, as it is often the case with these two tissues. Nevertheless, according to manufacturer, these variations in GAPDH expression reflect a tissue-specific expression rather than a non-equal loading of the samples.

Specifically, FIG. 2 shows the tissue distribution of RAP250 mRNA.

In FIG. 2A, a human multiple tissue Northern blot (Clontech) containing 2 μg of poly (A+) RNA from each tissue was probed with ³²P-labeled RAP250 and GADPH (control) cDNAs. Northern blotting revealed major approximate 7.5 kb RAP250 transcript, and this messenger was differentially transcribed among the various tissues. Two alternative transcripts of different sizes are also present in peripheral blood leukocyte (approx. 6 kb) molecular weights are indicated on the left in kilobases.

FIG. 2B shows RAP250 mRNA expression in mouse embryo (Ba-Bc), postnatal and adult mouse tissues (Bd) and adult rat tissues (Be-Bh). (Ba) E9 mouse embryo: neural epithelium (ne), placenta (pl). (Bb) E15 mouse embryo: developing neocortex (co), thalamus (th), basal ganglia (bg), cerebellum (ce), spinal cord (sc), olfactory epithelium (oe), whisker follicles (wf), thymus (th) , heart (he), liver (li), intestine (in), kidney (ki). (Bc) 1.5 day old mouse : hippocampus (hc), tooth (to), salivary gland (sg), lung (lu), stomach (st), spleen (sp), skin (sk). (Bd) 8 day old mouse eye: retina (arrowheads), lens (le). (Be) Rat testis: seminiferous tubules (arrowheads). (Bf) Rat prostate. (Bg) Rat ovary: follicles (arrowheads), interstitial tissue (it), corpus luteum (cl). (Bh) Mouse brain: cerebral cortex (co), hippocampus (hc), piriform cortex (pco).

5) In Situ Hybridisation

Adult male and female Sprague-Dawley rats, NMRI mice and postnatal mice (1.5, 4, 8 and 14 days) were decapitated and the tissues were excised and frozen on dry ice. Embryonic mice (e9-e17) and rats (e12-e21) were excised from pregnant females and frozen. The tissues were sectioned with Microm HM-500 cryostat at 14 μm and thawed on Polysine glasses (Menzel, Germany). In situ hybridization was carried out as previously described (Kononen, J., et al (1997) Trends Genetic. Technical Tips Online: http/tto.trends.com/). Two oligonucleotide probes directed against the mouse RAP250 mRNA (nucleotides 3185-3219 and 3448-3479 as numbered on human sequence) were used. The sequences had 100% (3185-GCACCCCCACCACAGCCACCACAGCAGCAGCCACA-3219) and 96% (3448-GCAAGGACCTGCCTCTGTGCCACCATCACCTG-3479) homology to human RAP250 and less than 70% homology with any other known gene compared to the known sequences in the GenBank database. Both probes produced similar results when used separately and were usually combined to intensify the hybridization signal. Several probes to non-related mRNAs with known expression patterns, with similar length and GC-content, were used as controls to verify the specificity of the hybridizations.

6) In Vitro Translation of RAP250 cDNA

The human full-length RAP250 CDNA cloned in pSG5 was in vitro transcribed and translated using rabbit reticulocyte lysate (TNT coupled in vitro system Promega) according to manufacturer's recommendations with [₃₅S] methionine. The radiolabeled protein was separated by 7% SDS-PAGE and the gel was then dried and autoradiographed.

7) Expression and Purification of GST-Fusion Proteins

Cultures of E. Coli BL21(pLys) carrying the pGEX fusion constructs (RAP250, ERα, TRα) were grown at 37° C. in Luria-Bertani medium containing 100 μg/ml ampicilline and 34 μg/ml chloramphenicol and supplemented with 2% glucose. At OD₆₀₀ 0.6 the cultures were induced with 0.5 mM IPTG (isopropyl-β-D-thiogalactopyranoside) for 2 to 3 h at 30° C. GST and GST-RAP250 proteins were purified as described previously (Treuter, E., et al, supra) and the bacteria expressing GST fused to nuclear receptors were harvested by centrifugation and resuspended in STE-buffer (10 mM Tris-HCl [pH8], 150 mM NaCl, 1 mM EDTA) and frozen on dry ice. After thawing, lysozyme was added to a final concentration of 0.1 mg/ml and the suspension rotated at 4° C. for 15 min. Then DTT was added to a final concentration of 5 mM and Sarcosyl to 1.5%. After centrifugation at 10,000×g for 30 min at 4° C. the lysates were added to glutathione-Sepharose 4B (Amersham Pharmacia Biotech) for 2 h at 4° C. and washed three times with phosphate-buffered saline (PBS). To produce pure GST fusion protein for electrophoretic mobility shift assay (EMSA), the proteins were eluted with 4 volumes of 20 mM glutathione in 50 mM Tris-HCl [pH8]. Protein concentrations were determined by the Bradford dye-binding procedure (Bio-Rad Laboratories).

8) In Vitro Protein-Protein Interaction Assay (GST Pull-Down Assay)

All the nuclear receptors we tested in pull-down assays were in vitro transcribed and translated using rabbit reticulocyte lysate (TNT coupled in vitro system Promega) according to the manufacturer's recommendations with [₃₅S] methionine. Approximately 5 μg of GST-fusion protein bound to glutathione-Sepharose 4B beads (Amersham Pharmacia Biotech) was used in each assay. The beads were incubated for 3 h with rotation at +4° C. with 2 μl of [₃₅S] methionine-labeled protein in the presence of 2 μl of the appropriate ligand in dimethyl sulfoxide (DMSO) or ethanol at final concentrations of 100 nM 9-cis RA [RXR], 100 μM Wy14643 [PPARα], 200 nM T₃ [TRs], 1 μM E₂ [ERs], 100 μM BRL [PPARγ] or vehicle alone in a total volume of 200 μl of incubation buffer (20 mM Hepes KOH [pH 7.9], 20% (v/v) glycerol, 100 mM KCl, 5 mM MgCl₂, 0.2 mM EDTA, 0.01% NP-40, 1.5% bovine serum albumin (BSA), 1 mM DTT, 0.2 mM PMSF) supplemented with a protease inhibitor cocktail (Complete, Boehringer Mannheim). Beads were separated by centrifugation (2000 g) and washed three times for 15 min with incubation buffer without BSA. Washed beads were resuspended in 60 μl of 1×sodium dodecyl sulphate (SDS) sample buffer, and an aliquot was subject to SDS-polyacrylamide gel electrophoresis. Before autoradiography, gels were stained with Coomassie blue to ensure the stability of the GST fusion proteins and equal loading of the samples in the wells provided.

Specifically, FIG. 3 shows the results on the in vitro interaction between mRAP250 and TRs, PPARs, and ERs.

A, Schematic representation of GST protein fused to wild type (wt) and mutated (mut) NR-box containing fragments of mRAP250 used in pull down assay with in vitro translated NRs.

B, Addition of ligand enhances RAP250 interaction with PPARs, TRs, and ERs. The nuclear receptors were in vitro translated in the presence of [₃₅S] methionine and analysed for interaction with GST, GST-wt RAP250 or GST-mut RAP250 bound to glutathione-Sepharose beads in a pull-down assay. The interaction was analysed in presence(+) or absence (−) of the corresponding ligand. Final concentrations of ligands were 100 nM 9-cis RA [RXR], 100 μM Wy14643 [PPARα], 200 nM triiodothyronine (T₃) [TRs], 1 μM estradiol (E₂) [ERs], 100 μM BRL [PPARγ].

Mutation of LVNLL motif to AVNAL completely abolished RAP250 interaction with NRs. 1: input, 2: GST, 3: GST-wt RAP250 without ligand, 4: GST-wt RAP250 with ligand, 5: GST-mut RAP250 without ligand, 6: GST-mut RAP250 with ligand. Input represents 50% of the amount of labeled protein used in the pull-down assay.

9) Electrophoretic Mobility Shift Assays

TRα, and RXRα, were synthesized in rabbit reticulocyte lysate by using the TNT coupled in vitro transcription-translation system (Promega). Double-stranded oligonucleotides synthetic DR4-TRE 5′-TCGATCAGGTCATTTCAGGTCAGAG-3′ were radioactively-labeled with [(α-₃₂P]dCTP. Binding reactions were performed in a total volume of 20 μl in 1×reaction buffer [5% glycerol, 5mM DTT, 5 mM EDTA, 250 mM KCl, 100 mM HEPES (pH 7.5), 1 μg of poly(dIdC), 25 mM MgCl₂, 1 mg BSA per ml, 1 μg salmon sperm DNA, 0.05% Triton-X100], 0.5 ng of labeled probe, 2 μl of each in vitro-translated receptor protein, and when indicated, 2 μl of appropriate ligand in DMSO. Finally, 0.5 μg per reaction of the purified GST-RAP250 was added as indicated in results. The binding reaction was allowed to proceed for 20 min on ice before the reaction mixtures were loaded on a 4% non-denaturing polyacrylamide gel. After 3 h electrophoresis in 0.5×Tris-Borate-EDTA (TBE) buffer at 4° C., the gels were dried and autoradiographed.

10) Cell Cultures and Transient Transfections

COS-7 cells were maintained in Dulbecco's modified Eagle's medium (DMEM) supplemented with 10% FCS, 100 μg/ml penicillin and 100 μg/ml streptomycin (Life Technologies Inc.). For transient transfection assays, COS-7 cells were plated onto 6-well plates (Falcon) 24 h prior to transfection. Cells were transfected using Lipofectin as instructed by the manufacturer (Life Technologies Inc.). For each well 0.5 μg reporter plasmid and 0.5 μg of Gal4 expression plasmid were transfected. 24 h after transfection cells were harvested and cell extracts were analyzed for luciferase activity as described (Treuter. E., et al, supra).

In the mammalian interaction and/or coactivation assays, 100 ng wild type NR plasmids (TRs, RXR, PPARs) and 0.5 μg of VP16 or VP16-mRAP250 plasmid (interaction assay) or 0.5 μg of pSG5 or pSG5-hRAP250 plasmid (coactivation assay) were transfected into COS7 cells together with 0.5 μg appropriate reporter plasmid (DR4-tk-Luc, PPRE-tk-Luc, DR1-tk-Luc and ERE-TATA-Luc as previously mentioned). Transfections were carried out for 4 to 6 h and the cells were then grown in presence of appropriate ligand or DMSO for 36 h and then harvested and analyzed as previously described (Treuter. E., et al, supra). Transfections with wild type ERα and β were carried out using 293 cells (human kidney cell line from A.T.C.C.). 293 cells were plated on 6-well plates containing phenol red-free Dulbecco's medium supplemented with 10% charcoal stripped FCs, 100 μg/ml streptomycin (Life Technologies Inc.).

11) GeneBank Accession Numbers

The protein and nucleotide human sequences of RAP250 have been submitted to EMBL/GenBank data base with accession number AF128458. The partial mouse nucleotide and protein sequences of RAP250 have been submitted to EMBL/GenBank database with accession number AF135169.

12) Human RAP250 is Ubiquitously Expressed with a Major Expression in Reproductive Organs and Brain

Northern blot analysis of human RNAs revealed a widespread major RAP250 transcript of approximately 7.5 kb in length, which was expressed at different levels depending on the tissue (FIG. 2A). High expression was detected in reproductive organs such as ovary, testis and prostate, as well as in peripheral blood leukocytes, brain and heart, and an intermediate expression was observed in pancreas, kidney, liver, colon, spleen and placenta. Expression of RAP250 was low but still detectable, in small intestine, thymus, and skeletal muscle. Among the human tissues rich in RAP250 mRNA, ovary, testis, prostate and brain cells are known to express high levels of at least one of the two estrogen receptor subtypes (ERα and ERβ). Taken together these data indicated that, although widely expressed in humans, the highest levels of RAP250 are found in ER rich tissues. Interestingly, in testis, a second transcript of approximately 4.5 kb in length was also highly expressed. Subcloning and sequence analysis of this shorter messenger indicated that it was an alternatively spliced form of RAP250 with an open reading frame encoding a 1071 aminoacid-protein (data not shown). Thus, considering the expression of both isoforms, testis appears to be the main RAP250 expressing organ. In peripheral blood leukocytes, a second 6 kb transcript was also detected which might represent either another alternatively spliced form or a closely related, but different isotype.

13) From Widespread Embryonic Expression of RAP250 RNA to More Restricted Expression in Adult Tissues

Overall expression of RAP250 mRNA in mouse and rat embryos was quite similar. RAP250 mRNA was widely expressed during ontogeny. At e9, clear signal was present in placenta and lower expression could be seen in uterus (FIG. 2Ba). At this stage, neural tube expressed high levels of RAP250 mRNA and the expression in central nervous system was high throughout the ontogeny (FIG. 2Ba-c). The expression in spinal cord and in cerebrum was high and became more restricted during later stages of development (e17 onwards) and postnatal life. High expression was seen in cerebellum during the period of cerebellar development (FIG. 2Bc). Also sensory ganglia and retina showed high expression from e11 onwards (FIG. 2Bd). In alimentary tract (oral cavity, stomach and intestine) expression was seen from e13 and throughout the ontogeny (FIG. 2Bb-c). The developing teeth and salivary gland were also labelled (FIG. 2Bc). Olfactory epithelium was strongly labelled from e13 onwards (FIG. 2Bb-c). Strong expression was present in liver (from e11) and in kidney (from e13 onwards) and levels decreased at later stages of development (FIG. 2Bb-c). Lungs had moderate signal from e13 and the levels decreased during postnatal life (FIG. 2Bc). Prominent signal was seen in thymus from e15 onwards, and in spleen from e17 and during early postnatal life and subsequently the expression decreased (FIG. 2Bb-c). Low to moderate signal was seen in brown fat as well as developing muscles, bones and intervertebral discs. In adult mouse and rat, expression of RAP250 mRNA was more restricted than during embryonic development. High expression was nevertheless observed both in male and female rat genital organs. In testis, seminiferous tubules exhibited a strong signal and the expression in separate tubules varied, indicating that RAP250 is expressed in a stage-specific manner during spermatogenesis (FIG. 2Be). In dipped sections, RAP250 mRNA could be seen in primary spermatocytes. Prominent expression was also seen in the epithelium of prostate, while epididymis and vesicula seminalis had low signal (FIG. 2Bf). In ovary, the strongest signal was seen in interstitial cells and in the granulosa cells of different size follicles (FIG. 2Bg). In central nervous system, high expression was present in olfactory bulb, piriform cortex, hippocampus and cerebellar cortex, while other areas exhibited lower levels of RAP250 mRNA (FIG. 2Bh). In the liver, heart and kidneys the expression was low (data not shown).

Two Isoforms of RAP250 are Coexpressed in Human Testis

To detect the endogenous RAP250 protein and to determine its native size, a Western blot analysis was performed (as previously described) using a polyclonal RAP250 antibody. Furthermore, since Northern blot analysis revealed that two RAP250 transcripts were present in human testis (FIG. 2A), human testis whole homogenates were used in order to determine if the two mRNAs were both functional and translated into two different proteins. As shown in FIG. 2C, a short RAP250 isoform of approximately 120 kDa was strongly expressed in human testis. The size of this protein indicates that it is very likely encoded by the 4.5 kb transcript present in human testis (FIG. 2A). In addition, human testis also expressed a 220 kDa protein, although at a lower level. The size of this bigger protein is consistent with the calculated molecular weight of the protein encoded by the open reading frame of RAP250 (2063 amino acids), These results indicate that the 4.5 kb mRNA, which is a splicing variant of the longer 7.5 kb MRNA (data not shown), is a functional mRNA that encodes a second RAP250 protein in the testis. The reason why the testis coexpresses both RAP250 proteins is not yet known.

14) RAP250 Interacts With PPARs, TRs and ERs In Vitro Via the LxxLL Motif

The partial mouse RAP250 clone was originally isolated via its interaction with the PPARα-LBD bait in the yeast two-hybrid system. To determine if the mouse RAP250 can also interact with other nuclear receptors, we set up an in vitro protein-protein interaction assay with the mouse RAP250 clone (aa 782-1138) fused to GST, and referred to as GST-RAP250, and radioactively labelled in vitro translated nuclear receptors. As shown in FIG. 3, PPARs, TRs and ERs specifically interact with GST-RAP250 but not with GST. The addition of appropriate ligands in the binding buffer increased the interaction between GST-RAP250 and most of NRs (FIG. 3B, lanes 3 and FIG. 4), indicating a ligand-dependent interaction in the cases of TRs, and a ligand enhanced-interaction for ERs and PPARs. To investigate if the LxxLL motif (LVNLL, aa 891-895) of mouse RAP250 was responsible for the interaction with nuclear receptors, the leucine core motif was mutated to AVNAL as shown in FIG. 3A. Mutation of this motif abolished the interaction of GST-RAP250 with all tested NRs both in the absence or presence of ligands (FIG. 3, lanes 5 and 6), indicating that the interaction between RAP250 and NRs was mediated by an LxxLL motif whose integrity is required to function as an NR-box.

15) RAP250 Contains Only One Functionally Active NR-Box

While the NR-interacting RAP250 mouse fragment contains only one NR-box domain which is identical between mouse and human proteins (see FIG. 1C), analysis of the human RAP250 protein sequence revealed a second LxxLL motif, (LSQLL, aa 1491-1495), that might possibly serve as an additional NR-box. To test this and further determine if other regions of the full-length RAP250 possibly interact with nuclear receptors, eight overlapping cDNA fragments covering the human RAP250 sequence (FIG. 4A) were in vitro translated and used in a GST pull-down assay with ERα or TRα expressed as GST fusion proteins, both in absence or presence of appropriate ligand.

The results are shown in FIG. 4 as follows:

A, Linear diagram of the RAP250 deletion constructs used in this study.

B, Analysis of interaction between 8 deletion constructs of RAP250 and GST-fused nuclear receptors: ERα and TRα. RAP250 truncated mutants were in vitro translated in the presence of [₃₅S] methionine and analysed with GST or GST-NR protein bound to glutathione-Sepharose beads in a pull-down assay. The numbers at the bottom of the figure indicate the location of each fragment in the RAP250 protein. Input represents 50% of the amount of labelled protein used in the pull-down assay. When indicated, appropriate ligand was added to the reaction volume at final concentration of 1 μM (E₂), 0.5 μM (T₃). The same pattern of interaction was observed for both NRs:only the RAP250 fragment containing the first LxxLL motif (LVNLL) showed a strong interaction with GST-NRs.

As seen in FIG. 4B, only fragment 4 (aa 819-1096) containing the first LxxLL motif interacts strongly with nuclear receptors whereas no interaction was observed with fragment 6 (aa 1491-1495) containing the second LxxLL motif, indicating that it is not functionally active as an NR-box. Its sequence, SLSQLL, does not closely fit the consensus motif largely found in coactivators which often contains a hydrophobic amino acid upstream of the first conserved leucine residue. This sequence difference might explain why this second motif is not functionally active as an NR-box. With the exception of fragment 5 (aa 1061-1338) that shows a very weak interaction with ERα, but not with TRα, none of the other RAP250 fragments interacts with NRs. However, the strength of the interaction between fragment 4 and ER is many-fold higher and this NR-box containing region should so far be considered as the interacting region. As previously observed in FIG. 3, RAP250 interacts in a ligand-dependent manner with TRα and in a ligand-enhanced manner with ERα. Taken together, these data indicate that the human RAP250, although containing two LxxLL motifs, only possesses one NR-interacting-domain containing a single functional NR-box.

FIG. 9. Schematic representation of RAP250. Shown on this scheme are the NR-box (black box), as well as two glutamine-stretches (double underlined) surrounding the activation domain represented as a stripped region. The glutamine-rich region includes the entire activation domain.

16) RAP250 Interacts with NRs Bound to DNA

To determine if RAP250 could interact with NRs bound to their DNA response elements we performed electrophoretic mobility shift assays (EMSA). Ternary complex formation with NR heterodimers was assessed using in vitro translated TRα/RXRα and the purified mouse RAP250 (aa 818-931) fused to GST. As seen in FIG. 5A, GST-protein alone does not interact with DNA bound TRα/RXRα, either in the presence or absence of ligands (FIG. 5A, lanes 1 and 2). Addition of GST-RAP250 to the binding reaction resulted only in a weak supershift in the absence of ligands (FIG. 5A, lane 3). However, addition of ligands, T3 or 9-cis RA or both, resulted in a pronounced mobility shift of the TRα/RXRα dimer (FIG. 5A, lanes 4-6), indicating the formation of an oligomeric complex containing GST-RAP250.

To further characterize the stoechiometry of this interaction we used helix 12 mutated forms of TRα and RXRα. These mutants have previously been shown not to interact with coactivators (Wiebel, F. F. et al, supra; Saatcioglu, F. et al, supra). When the mutated RXRα was used, the ligand dependent interaction of GST-RAP250 in response to 9-cis RA was lost (FIG. 5A, lane 11). However, GST-RAP250 still interacts with the NR heterodimer in presence of T₃ (FIG. 5A, lane 10 and 12). In a similar manner, when the mutated form of TRβ was used, the ligand dependent interaction of GST-RAP250 in response to T₃ was lost (FIG. 5B, lane 4) but GST-RAP250 still interacts with the NRs in response to 9-cis RA (FIG. 5B, lanes 5 and 6). When the mutated receptors are used in combination, receptor heterodimers are still detected on DNA but no interaction of GST-RAP250 can be detected (FIG. 5B, lanes 7-12). These results demonstrate that RAP250 is able to interact with DNA-bound NRs in a ligand and AF-2 dependent manner. Since the oligomeric complexes detected with GST-RAP250 and wild type receptors are located at the same position as the ones detected when one NRs is mutated this could suggest that only one GST-RAP250 molecule binds per receptor heterodimer.

17) RAP250 Differentially Interacts with Wild Type NRs in Mammalian Transfected Cells

In previous in vitro experiments, we demonstrated that RAP250 interacts with NRs in a ligand-dependent or a ligand-enhanced manner, depending on the NR. To determine if RAP250 interacts with NRs in vivo, we used a mammalian transient transfection assay derived from the two-hybrid assay, using VP16 activation domain fused to the mouse RAP250 clone (aa 782-1138). VP16-RAP250 expression vector or VP16 vectors were transfected into COS-7 cells together with expression vectors for wild type NRs and a luciferase reporter gene containing appropriate response elements. As seen in FIG. 6, TRα/RXRα-mediated activity of the reporter gene was 3.8-fold stimulated by VP16-RAP250, as compared to VP16. Similar stimulation was observed for PPARγ/RXRα, (3.3 fold), whereas RXRα-mediated reporter gene expression was stimulated 10.5 fold. These in vivo data support the observation made in vitro (FIG. 3) that RAP250 interacts with all tested nuclear receptors apparently with a different strength depending on the receptor.

18) RAP250 Activates Transcription via a Large Intrinsic Transcription Activation Domain

In order to identify activation and/or repression domains within RAP250, fragments of RAP250 were fused to the Gal4-DBD and analyzed for transcription activation potential using transient transfection assays in COS-7 cells. In a first set of experiments, eight Gal4-RAP250 constructs containing about 300 amino acids each were assayed for their putative transcriptional activity in mammalian COS-7 cells (FIG. 7A). Two of the constructs were able to activate transcription and none of them had any significant repression activity. The construct containing amino acids 335-630 activated transcription approximately 5-fold as compared to Gal4-DBD alone, and the construct containing amino acids 577-855 activated transcription about 10-fold. Since these two fragments partially overlapped each other, a second set of constructs was made to determine if there are two independent activation domains or if the two fragments have one common activation domain in the overlapping region. Removal of 52 amino acids from the Gal4-RAP250-(335-630) construct generated a Gal4-RAP250-(335-577) construct that reduced the activation capacity from 5-fold to 3-fold as shown in FIG. 7B. In a similar manner, 52 amino acids were removed from the Gal4-RAP250-(577-855) construct, generating a Gal4-RAP250-(630-855) which reduced the activation capacity from 10-fold to 6-fold (FIG. 7B). Moreover, the fragment spanning from amino acid 577 to aa 630, that is overlapping in the first set of fusion constructs (2 and 3), retains a weak activation potential, about 2-fold. An additional construct that contains all sequences that showed activation potential, i.e. Gal4-RAP250-(335-855), activated transcription about 18-fold. From these data we conclude that RAP250 contains one large activation domain localized between amino acids 335-855 and that removal of sequences within this activation domain gradually reduces its strength. The activation domain of RAP250 was also subcloned into the pGBT9 yeast expression vector in order to test if it could activate transcription in yeast. However, no activation was detected based on the β-galactosidase expression (data not shown), suggesting that it is inactive in yeast.

1) RAP250 Shows Preferential Coactivation with ERβ.

Since most of the identified coactivators can enhance the transcriptional activity of several nuclear hormone receptors (Shibata, H. et al (1997), Recent Prog. Horm. Res. 52:141-164), it was important to investigate whether RAP250 would function as a general coactivator of the transcriptional activity of various receptors or if it would be a more specific coactivator. This hypothesis was based on the fact that RAP250 exhibits some in vivo and in vitro binding preferences for some of the tested nuclear receptors.

As shown in FIG. 8, RAP250 further induced the transcriptional activity of all tested receptors both in absence and presence of appropriate ligands. However, among all the tested nuclear receptors, RAP250 showed the highest specificity to ERβ with a 27-fold enhancement of ERβ-mediated transactivation in presence of estradiol (3.8-fold in the absence of E₂), which is twice as strong as for ERα (13.3-fold activation) (FIG. 8A). Enhancement of transcriptional activities of TR, PPAR and RXR by RAP250 was lower than for ERs, although significant, being approximately 7.5-, 7-and 6-fold, respectively in the presence of ligand (FIG. 8B) and approximately 3-, 4-and 3-fold respectively, in the absence of ligand (FIG. 8B). It is indeed noteworthy that RAP250 enhanced the transcriptional activity of every nuclear receptor even in the absence of ligand, which is consistent with the in vitro observed interactions between the NRs and RAP250 and is probably due to the overexpression of RAP250. While RAP250 cannot be considered as a specific coactivator, these results nevertheless clearly indicate that this coactivator contributes preferentially to ERβ-mediated transcriptional activity.

In summary the inventors have disclosed the structural and functional properties of RAP250, a novel nuclear receptor coactivator isolated from a mouse embryo library using the yeast two-hybrid system. Based on sequence analysis, RAP250 appears to be different from other nuclear receptor coactivators characterized to date. For example, it shows no significant sequence homology with any known nuclear receptor coactivator, has no bHLH/PAS domain as found in the p160 proteins, or any motifs that would suggest histone acetylase or deacetylase activity.

Nevertheless, some specific features such as the presence of LxxLL motifs and a glutamine-rich activation domain classify this protein as a putative AF-2 coactivator (FIG. 9). Of the two LxxLL motifs found in RAP250 (FIG. 9), only the first one functions as an NR-box in contrast to the p160 proteins that contain multiple NR-boxes. In that respect, RAP250 resembles coactivators such as TIF-1 (Le Douarin, B. et al, (1995), EMBO J. 14:2020-2033; Thenot, S. et al (1997), J. Biol. Chem. 272:12062-12068) or PGC-1 (Puigserver, P. et al (1998), Cell 92:829-839) that also have only one functional NR-box.

One model of coactivators binding to NR-heterodimers proposes a bridging function of the p160 proteins by simultaneous binding of the two dimer-subunits with two adjacent NR-boxes (Westin, S. et al (1998), Nature 395:199-202). However, this possibility clearly does not exist for RAP250 having only one NR-box. Another model, suggested by the presence of only one functional NR-box, could involve two molecules of coactivator per NR-dimer, each of the subunits of the dimer binding one coactivator molecule via the NR box (Leers, J. et al (1998), Mol. Cell. Biol. 18:6001-6013).

The inventors have provided evidence that the interaction of RAP250 with ERs (as with other tested NRs) only involves one functional NR-box within the entire protein. Recent data suggests that there is a functional in vivo requirement for only one of the three NR-boxes of SRC-1 for ER-interaction and activation (McInerney, E. M. et al (1998), Genes & Dev. 12:3357-3368). The presence of only one functional NR-box in RAP250 could be consistent with these stoichiometric data, each of the subunits of the ER-dimer binding one RAP250 molecule via the NR box. Alternatively, RAP250 could be part of a larger complex of proteins, in which it would bind a nuclear receptor via its NR-box, and another subunit of the complex would bind the other NR-dimer subunit. Such a coactivator complex, called TR-associated multiprotein (TRAP) complex, composed of several different proteins, among which TRAP220 is the NR-binding subunit, and exhibiting an in vitro transcriptional coactivator function has recently been identified [is there a reference for this work?]. It would be interesting to determine if RAP250, as TRAP220, could bind to NR-dimers in a similar complex.

Based on functional analyses, RAP250 behaves as some coactivators of the p160 family. In particular, it has been shown that RAP250 enhances, in a ligand-dependent manner, the transcriptional activity of multiple members of the nuclear receptor family, indicating that RAP250 is a coactivator of all these NRs. In the present study, we also provide evidence that RAP250 and TIF2 efficiently compete for binding to ERβ (FIG. 5D) and to TRs (data not shown). Therefore, RAP250 may act not only as coactivator but also as a coregulator by competition with other coactivators for binding to receptor dimers. While competition might be relevant for NR-box mediated interactions, the possibility of simultaneous binding of NR-box dependent cofactors with NR-box independent cofactors such as TRAP230 or P/CAF should also be considered. Such a scenario would provide another mechanism for receptor specificity and formation of alternative coactivator complexes. In addition, RAP250 possesses an intrinsic transcriptional activation domain and functions as a nuclear receptor coactivator in mammalian cells. This activation domain is large and rich in glutamine with approximately 20% of Q residues (FIG. 1A and 9), but is not active when tested in yeast (data not shown). This is in agreement with previous findings demonstrating that glutamine-rich activation domains of transcription factors Oct-1, Oct-2 and Sp1, which activate transcription in mammalian cells are inactive in yeast, probably reflecting some basic difference between the organization of yeast and mammalian promoters or transcription complexes (Künzler, M. et al (1994), EMBO J. 13:641-645). Interestingly, the enhancer-binding protein Sp1, which is a prototype for glutamine-rich transcription factors, was recently shown to interact with a transcription complex called CRSP which contains TRAP220 (Ryu, S. et al (1999), Nature 397:446-450).

In aspects such as expression pattern and NR-specificity, RAP250 differs from the p160 coactivators. First, although widely expressed in human cells, RAP250 shows a more specific expression than p160 proteins, with a high expression in brain and reproductive organs such as ovary, testis, and prostate (FIG. 2A), known as major target organs for estrogen action (Kuiper, G. G. et al (1997), Endocrinology 138:863-870). This pattern of expression suggests that RAP250 might be involved in the regulation of tissue-selective gene expression. However, it should be mentioned that RAP250 is also expressed in the spleen where no noticeable levels of ER were detected (Kuiper, G. G. et al (1997), Endocrinology 138:863-870). However, the spleen has a very large content of PPARγ (Braissant, O. et al (1996), Endocrinology 137:354-366) which also interacts with RAP250, so co-localization of these two proteins could be of physiological relevance. Secondly, in both binding and coactivation experiments, RAP250 shows specific preferences for ERβ and might function as a preferential coactivator for ERβ-mediated transactivation. Based on the amino acid differences within the AF-1 and AF-2 domains of ERα and ERβ and on the ligand and response element, it has been suggested that the regulation of ERα and ERβ responsive genes may use different molecular mechanisms, including the use of distinct coactivators (Suen, C. S. et al (1998), J. Biol. Chem. 273:27645-27653; Paech, K. et al (1997), Science 277:1508-1510).

Thus, the experiments previously described reveal an unexpected feature of RAP250 in its differential interaction with ERα/β, and it appears that RAP250 is the first nuclear receptor AF-2 coactivator displaying such a ER dimer selectivity. Although p160 coactivators may exhibit different affinities to certain receptors, they do not distinguish between different receptor dimers. In that context, it is worth considering the possible contribution of the F-domain which is not conserved between the two ERs. Mutational analysis will be required to clarify whether the NR-box itself or adjacent regions are responsible for the ER dimer selectivity. Although the reasons for these differences between ERα and β subtypes are as yet unclear, these observation raise the possibility that RAP250 could have different effects on transcriptional regulation mediated by the two ER subtypes, that are co-expressed in certain tissues. In conclusion, the inventors have identified and characterized a novel coactivator that may represent one of the first identified ER-coactivators with ER subtype-specificity.

19) RAP250 Functions as a Coactivator for Nuclear Receptor

Based on the fact that RAP250 binds in vivo and in vitro with all the tested nuclear hormone receptors, it was important to investigate whether RAP250 would also function as a coactivator on the transcriptional activity of these receptors. To investigate this, expression vectors for TRα and RXRα were cotransfected with pSG5-hRAP250 or the empty vector together with a luciferase reporter gene, DR4-Tk-Luc for TRα or DR1-Tk-Luc for RXRα. As shown in FIG. 8, addition of appropriate ligand alone induced the expression the reporter genes 4.6-fold for TRα/RXRα and 3.2-fold for RXR. Overexpression of hRAP250 further induced the expression to 7.2-fold and 7.7-fold respectively. Thus, these results show that RAP250 can act as a coactivator.

The invention comprises the structural and functional properties of RAP250, a novel nuclear receptor coactivator isolated from a mouse embryo library using the yeast two-hybrid system. Our results show that RAP250 interacts with multiple members of the nuclear receptor family in a ligand-dependent manner, indicating that RAP250 is a coactivator of all these NRs. We also show that this interaction is dependent on an NR-box and an intact AF-2 domain. Furthermore, RAP250 possesses an intrinsic transcriptional activation domain and functions as a nuclear receptor coactivator in mammalian cells. A schematic representation of RAP250 is shown in FIG. 9. Together these results show that RAP250 is a new AF-2 NR coactivator. Based on sequence analysis, RAP250 appears to be different from other nuclear receptor coactivators characterized to date. For example, it shows no significant sequence homology with any known nuclear receptor coactivator, has no bHLH/PAS domain as found in the p160 proteins, or any motifs that would suggest histone acetylase or deacetylase activity.

Out of the two LxxLL motifs found in RAP250, only the first one functions as an NR-box in contrast to the p160 proteins that contain multiple NR-boxes. In that respect, RAP250 resembles coactivators such as TIF-1 (Le Douarin, B., et al (1995) EMBO J. 14: 2020-2033; Thénot, S., et al (1997) J. Biol. Chem. 272: 12062-12068) or PGC-1 (Puigserver, P., et al (1998) Cell 92: 829-839) that also have only one functional NR-box. One model of coactivators binding to NR-heterodimers proposes a bridging function of the p160 proteins by simultaneous binding of the two dimer-subunits with two adjacent NR-boxes (Westin, S., et al (1998) Nature 395: 199-202). However, this possibility clearly does not exist for RAP250 having only one NR-box. Another model, suggested by the presence of only one functional NR-box, could involve two molecules of coactivator per NR-dimer, each of the subunits of the dimer binding one coactivator molecule via the NR box (Leers, J. et al, supra). In the case of RAP250, it would mean that each of the subunits of the NR-dimer binds one RAP250 molecule via the NR box. However, the EMSA analysis (FIG. 5) does not suggest that this is the case for RAP250 since the complexes containing RAP250 and wild type NRs migrate at the same speed as when one of the NRs in the dimer has a mutation that prevents the coactivator to bind. An alternative could be that RAP250 is part of a larger complex of proteins, in which it would bind a nuclear receptor via its NR-box, in a similar manner as TRAP220/DRIP205 in the TRAP/DRIP complex (Fondell, J. D., et al, supra; Fondell, J. D., et al, supra; Treuter, E., et al (1999) J. Biol. Chem. 274: 6667-6677; Yuan, C. X., et al (1998) Proc. Natl. Acad. Sci. USA 95: 7939-7944). Since RAP250 does not correspond to any of the subunits that have been identified (Ito, M., et al (1999) Mol. Cell. 3: 361-370; Rachez, C., et al (1999) Nature 398: 824-828), it is possible that RAP250 either is part of a new coactivator complex or represents an unidentified member of the TRAP/DRIP complex.

The RAP250 activation domain is large and glutamine-rich with approximately 20% of Q residues (FIG. 1A and 9), but is not active when tested in yeast (data not shown). This is in agreement with previous findings demonstrating that glutamine-rich activation domains of transcription factors Oct-1, Oct-2 and Sp1 which activate transcription in mammalian cells, are inactive in yeast, probably reflecting some basic difference between the organization of yeast and mammalian promoters or transcription complexes (Künzler, M., et al (1994) EMBO J. 13: 641-645). Interestingly, the enhancer-binding protein Sp1, which is a prototype for glutamine-rich transcription factors, was recently shown to interact with a transcription complex called CRSP, which contains TRAP220 (Ryu, S., et al (1999) Nature 397: 446-450).

RAP250 mRNA is widely expressed with apparently highest expression in brain and reproductive organs such as ovary, testis, and prostate (FIG. 2A and 2B).

1. An isolated mammalian coactivator, RAP250, the coactivator comprising the amino acid sequence of FIG. 1A and polypeptides, other than the polypeptide of FIG. 11A (KIAA0181), comprising at least a portion of the amino acid sequence of FIG. 1A and having a biological activity of RAP250.

2. An isolated mammalian coactivator, RAP250, the coactivator consisting of the amino acid sequence of FIG. 1A.

3. An isolated mammalian coactivator according to claim 1 or 2 and comprising 2063 amino acid residues.

4. An isolated mammalian coactivator according to claim 1, 2 or 3 and having a molecular weight of about 250 kDa.

5. A polypeptide according to claim 1 and having at least 70% sequence identity to the sequence of FIG. 1A.

6. A polypeptide according to claim 5 and having at least 80% homology to the sequence of FIG. 1A.

7. A polypeptide according to claim 6 and having at least 90% sequence identity to the sequence of FIG. 1A.

8. A polypeptide, other than the polypeptide of FIG. 11A (KIAA1181), having at least 50% identity to amino acids 819-1096 of FIG. 1A.

9. A polypeptide according to claim 8 and having at least 60% identity, to amino acids 819-1096 of FIG. 1A.

10. An isolated coactivator or polypeptide according to any preceding claim in which the biological activity is interaction with a nuclear receptor such as at least one of TR, RXRA, PPAR, LXR or an ER.

11. An isolated coactivator or a polypeptide according to claim 10 in which the interaction is binding with a nuclear receptor.

12. An isolated coactivator or a polypeptide according to claim 11 in which the nuclear receptor is in homo-or heterodimeric form.

13. An isolated coactivator or a polypeptide according to claim 10, 11 or 12 in which the nuclear receptor is bound to DNA.

14. An isolated coactivator or a polypeptide according to any preceding claim which binds ERβ more strongly than ERα.

15. An isolated coactivator or a polypeptide according to claim 14 which binds ERβ at least about twice as strongly as ERα.

16. An isolated coactivator or a polypeptide according to any preceding claim which displays ligand-dependent interaction with TR.

17. An isolated coactivator or a polypeptide according to any one of claims 1 to 16 which displays ligand-enhanced interaction with ER and PPAR.

18. An isolated coactivator or a polypeptide according to any preceding claim in which the biological activity is enhancement of transcription of at least one of TR, PPAR , ERα, Erβ, LXR and RXR.

19. An isolated coactivator or a polypeptide according to any preceding claim in which the biological activity is the ability to form a complex with DNA-bound nuclear receptor.

20. An isolated coactivator or a polypeptide according to claim 19 in which the nuclear receptor is selected from TR, PPAR ER, LXR and RXR.

21. A fusion protein comprising at least a portion of a polypeptide according to any preceding claim and a non-coactivator-related amino acid sequence.

22. An isolated coactivator or polypeptide according to any preceding claim and having at least one LXXLL amino acid motif.

23. An isolated coactivator or polypeptide according to claim 22 and having two or more LXXLL amino acid motifs.

24. Isolated coactivator or polypeptide according to claim 22 or 23 and having a single functional LXXLL amino acid motif.

25. An isolated coactivator or a polypeptide according to any preceding claim and having a single functional NR box.

26. An isolated coactivator or a polypeptide according to claim 25 in which the NR box includes a single functional LXXLL amino acid motif.

27. An isolated coactivator or a polypeptide according to any preceding claim and having an N terminal Q-rich region flanked by two poly-Q stretches.

28. An isolated mammalian coactivator according to any preceding claim which is derived from human cells.

29. An isolated mammalian coactivator according to any one of claims 1 to 27 which is derived from mouse cells.

30. An isolated mammalian coactivator according to any preceding claim which is expressed in brain and reproductive organ cells.

31. An isolated mammalian coactivator according to claim 30 which is expressed in humans at relatively high levels in at least one of reproductive organs such as ovary, testis or prostate; or peripheral blood leukocytes, brain and heart.

32. An isolated mammalian coactivator according to claim 30, or 31 which is expressed in humans at relatively moderate levels in at least one of pancreas, kidney, liver, colon, spleen, and placenta.

33. An isolated mammalian coactivator according to claim 30, 31 or 32 which is expressed in humans at relatively low levels in at least one of small intestine, thymus, and skeletal muscle.

34. A nucleotide sequence encoding a mammalian coactivator or polypeptide according to any preceding claim, other than the nucleotide sequence of FIG. 11B (KIAA181).

35. A nucleotide sequence according to claim 34 and comprising the DNA sequence of FIG. 1A or 10.

36. A nucleotide sequence according to claim 34 and consisting of the DNA sequence of FIG. 1A or 10 or fragments thereof; DNA sequences which encode a polypeptide having the same amino acid sequences encoded by FIG. 1A or 10 or a fragment thereof; and sequences which hybridise thereto under stringent conditions.

37. A nucleotide sequence according to claim 36 and comprising 6189 base pairs.

38. A vector including at least a portion of a nucleotide sequence according to any one of claims 34 to 37.

39. A method of isolating nuclear receptor coactivators comprising detecting binding of putative NR coactivators with at least one nuclear receptor.

40. A method according to claim 39 in which the nuclear receptor is selected from TR, ER, PPAR, and RXR.

41. A method according to claim 39 or 40 in which the binding is performed in the presence of a nuclear receptor ligand.

42. Probes for identifying coactivators comprising at least a portion of the sequence:
a. GCACCCCCACCACAGCCACCACAG CAGCAGCCACA
b. GCAAGGACCTGCCTCTGTGCCACCATCACCTG

43. Cells, tissues or organisms engineered to contain a nucleotide sequence according to any one of claims 34 to 37 or a vector according to claim 38.

44. Cells, tissues or organisms engineered to express a recombinant form of a coactivator or a polypeptide according to any one of claims 1 to 33.

45. A method of identifying nuclear receptor ligands, the method comprising testing the effect of a putative ligand on the interaction of an isolated coactivator or a polypeptide according to any preceding claim and a nuclear receptor.

46. A method according to claim 45 in which the interaction is binding.

47. A method according to claim 45 or 46 in which the nuclear receptor is an ER, TR, PPAR, LXR or RXR.

48. Nuclear receptor ligands obtainable by a method according to claim 45, 46 or 47.

49. A method of identifying selective estrogen receptor modulators (SERMs), in which the effect of a putative SERM on the interaction of an isolated coactivator or polypeptide according to any one of claims 1 to 32 with ERα and ERβ is tested.

50. A method according to claim 49 in which the interaction is binding.

51. ER ligands obtainable by a method according to any one of claims 45 to 50.

52. A method for screening for a compound which binds to a nuclear receptor coactivator or polypeptide according to any one of claims 1 to 33, the method comprising the steps of:
a) contacting the compound with a polypeptide having the amino acid sequence of FIG. 1B or a fragment thereof;
b) determining whether the compound specifically binds to the polypeptide, wherein the specific binding of the compound to the polypeptide identifies the compound as a compound which binds to the nuclear receptor coactivator.

53. A pharmaceutical composition comprising an isolated coactivator or a polypeptide according to any one of claims to 1 to 33 or a ligand or compound obtained or identified by a method according to claim 45, 46, 47, 49, 50 or 52.

54. A method of treating a nuclear receptor-related disorder or condition in a subject comprising supply the subject with a pharmaceutical composition according to claim 53.

55. A method according to claim 54 in which the nuclear receptor-related condition is selected from cancer of the breast or uterine cancer; endometriosis; cardiovascular conditions; hot flushes; psychological conditions, such as depression, mood; anti-inflammatory indications such as asthma, upper airway and osteoporosis.

56. A method of restoring wildtype RAP250 function in a subject, the method comprising supplying the subject with a nucleotide sequence according to claim 34, 35, 36 or 37 or a vector according to claim 38.

57. A coactivator having an amino acid sequence derived by expression of the nucleotide sequence of FIG. 10 or a portion of that sequence.

58. A coactivator according to claim 57 which is expressed in testis.

59. A coactivator according to claim 57 or 58 which is only expressed in testis.

60. An isolated mammalian coactivator of about 6 kilo base pairs and which is expressed only in peripheral blood leukocytes.
